SEQUENCE LISTING (170 sequences)

SEQ ID NO: 1 - DNA, 5386 bp, Bacteriophage phi-X174
cctgcagagttttatcgcttccatgacgcagaagttaacactttcggatatttctgatgagtcgaaaaattatcttgataaagcaggaattactactgcttgtttacgaattaaatcgaagtggactgctggcggaaaatgagaaaattcgacctatccttgcgcagctcgagaagctcttactttgcgacctttcgccatcaactaacgattctgtcaaaaactgacgcgttggatgaggagaagtggcttaatatgcttggcacgttcgtcaaggactggtttagatatgagtcacattttgttcatggtagagattctcttgttgacattttaaaagagcgtggattactatctgagtccgatgctgttcaaccactaataggtaagaaatcatgagtcaagttactgaacaatccgtacgtttccagaccgctttggcctctattaagctcattcaggcttctgccgttttggatttaaccgaagatgatttcgattttctgacgagtaacaaagtttggattgctactgaccgctctcgtgctcgtcgctgcgttgaggcttgcgtttatggtacgctggactttgtaggataccctcgctttcctgctcctgttgagtttattgctgccgtcattgcttattatgttcatcccgtcaacattcaaacggcctgtctcatcatggaaggcgctgaatttacggaaaacattattaatggcgtcgagcgtccggttaaagccgctgaattgttcgcgtttaccttgcgtgtacgcgcaggaaacactgacgttcttactgacgcagaagaaaacgtgcgtcaaaaattacgtgcagaaggagtgatgtaatgtctaaaggtaaaaaacgttctggcgctcgccctggtcgtccgcagccgttgcgaggtactaaaggcaagcgtaaaggcgctcgtctttggtatgtaggtggtcaacaattttaattgcaggggcttcggccccttacttgaggataaattatgtctaatattcaaactggcgccgagcgtatgccgcatgacctttcccatcttggcttccttgctggtcagattggtcgtcttattaccatttcaactactccggttatcgctggcgactccttcgagatggacgccgttggcgctctccgtctttctccattgcgtcgtggccttgctattgactctactgtagacatttttactttttatgtccctcatcgtcacgtttatggtgaacagtggattaagttcatgaaggatggtgttaatgccactcctctcccgactgttaacactactggttatattgaccatgccgcttttcttggcacgattaaccctgataccaataaaatccctaagcatttgtttcagggttatttgaatatctataacaactattttaaagcgccgtggatgcctgaccgtaccgaggctaaccctaatgagcttaatcaagatgatgctcgttatggtttccgttgctgccatctcaaaaacatttggactgctccgcttcctcctgagactgagctttctcgccaaatgacgacttctaccacatctattgacattatgggtctgcaagctgcttatgctaatttgcatactgaccaagaacgtgattacttcatgcagcgttaccatgatgttatttcttcatttggaggtaaaacctcttatgacgctgacaaccgtcctttacttgtcatgcgctctaatctctgggcatctggctatgatgttgatggaactgaccaaacgtcgttaggccagttttctggtcgtgttcaacagacctataaacattctgtgccgcgtttctttgttcctgagcatggcactatgtttactcttgcgcttgttcgttttccgcctactgcgactaaagagattcagtaccttaacgctaaaggtgctttgacttataccgatattgctggcgaccctgttttgtatggcaacttgccgccgcgtgaaatttctatgaaggatgttttccgttctggtgattcgtctaagaagtttaagattgctgagggtcagtggtatcgttatgcgccttcgtatgtttctcctgcttatcaccttcttgaaggcttcccattcattcaggaaccgccttctggtgatttgcaagaacgcgtacttattcgccaccatgattatgaccagtgtttccagtccgttcagttgttgcagtggaatagtcaggttaaatttaatgtgaccgtttatcgcaatctgccgaccactcgcgattcaatcatgacttcgtgataaaagattgagtgtgaggttataacgccgaagcggtaaaaattttaatttttgccgctgaggggttgaccaagcgaagcgcggtaggttttctgcttaggagtttaatcatgtttcagacttttatttctcgccataattcaaactttttttctgataagctggttctcacttctgttactccagcttcttcggcacctgttttacagacacctaaagctacatcgtcaacgttatattttgatagtttgacggttaatgctggtaatggtggttttcttcattgcattcagatggatacatctgtcaacgccgctaatcaggttgtttctgttggtgctgatattgcttttgatgccgaccctaaattttttgcctgtttggttcgctttgagtcttcttcggttccgactaccctcccgactgcctatgatgtttatcctttggatggtcgccatgatggtggttattataccgtcaaggactgtgtgactattgacgtccttccccgtacgccgggcaataatgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtcaaaaagccgcctccggtggcattcaaggtgatgtgcttgctaccgataacaatactgtaggcatgggtgatgctggtattaaatctgccattcaaggctctaatgttcctaaccctgatgaggccgtccctagttttgtttctggtgctatggctaaagctggtaaaggacttcttgaaggtacgttgcaggctggcacttctgccgtttctgataagttgcttgatttggttggacttggtggcaagtctgccgctgataaaggaaaggatactcgtgattatcttgctgctgcatttcctgagcttaatgcttgggagcgtgctggtgctgatgcttcctctgctggtatggttgacgccggatttgagaatcaaaaagagcttactaaaatgcaactggacaatcagaaagagattgccgagatgcaaaatgagactcaaaaagagattgctggcattcagtcggcgacttcacgccagaatacgaaagaccaggtatatgcacaaaatgagatgcttgcttatcaacagaaggagtctactgctcgcgttgcgtctattatggaaaacaccaatctttccaagcaacagcaggtttccgagattatgcgccaaatgcttactcaagctcaaacggctggtcagtattttaccaatgaccaaatcaaagaaatgactcgcaaggttagtgctgaggttgacttagttcatcagcaaacgcagaatcagcggtatggctcttctcatattggcgctactgcaaaggatatttctaatgtcgtcactgatgctgcttctggtgtggttgatatttttcatggtattgataaagctgttgccgatacttggaacaatttctggaaagacggtaaagctgatggtattggctctaatttgtctaggaaataaccgtcaggattgacaccctcccaattgtatgttttcatgcctccaaatcttggaggcttttttatggttcgttcttattacccttctgaatgtcacgctgattattttgactttgagcgtatcgaggctcttaaacctgctattgaggcttgtggcatttctactctttctcaatccccaatgcttggcttccataagcagatggataaccgcatcaagctcttggaagagattctgtcttttcgtatgcagggcgttgagttcgataatggtgatatgtatgttgacggccataaggctgcttctgacgttcgtgatgagtttgtatctgttactgagaagttaatggatgaattggcacaatgctacaatgtgctcccccaacttgatattaataacactatagaccaccgccccgaaggggacgaaaaatggtttttagagaacgagaagacggttacgcagttttgccgcaagctggctgctgaacgccctcttaaggatattcgcgatgagtataattaccccaaaaagaaaggtattaaggatgagtgttcaagattgctggaggcctccactatgaaatcgcgtagaggctttgctattcagcgtttgatgaatgcaatgcgacaggctcatgctgatggttggtttatcgtttttgacactctcacgttggctgacgaccgattagaggcgttttatgataatcccaatgctttgcgtgactattttcgtgatattggtcgtatggttcttgctgccgagggtcgcaaggctaatgattcacacgccgactgctatcagtatttttgtgtgcctgagtatggtacagctaatggccgtcttcatttccatgcggtgcactttatgcggacacttcctacaggtagcgttgaccctaattttggtcgtcgggtacgcaatcgccgccagttaaatagcttgcaaaatacgtggccttatggttacagtatgcccatcgcagttcgctacacgcaggacgctttttcacgttctggttggttgtggcctgttgatgctaaaggtgagccgcttaaagctaccagttatatggctgttggtttctatgtggctaaatacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggttacgacgcgacgccgttcaaccagatattgaagcagaacgcaaaaagagagatgagattgaggctgggaaaagttactgtagccgacgttttggcggcgcaacctgtgacgacaaatctgctcaaatttatgcgcgcttcgataaaaatgattggcgtatccaa

SEQ ID NO: 2 - DNA, 36 bp, Artificial Sequence
  staple sequence
gttcagaaaactggcctaaccactgcaacaagaaaa

SEQ ID NO: 3 - DNA, 50 bp, Artificial Sequence
  staple sequence
gcgtcacctgaaacaataagaggttttgaagaaataacatcatggtaacg

SEQ ID NO: 4 - DNA, 40 bp, Artificial Sequence
  staple sequence
aaagcggcatagatattcaaataagtacggtcaggcatcc

SEQ ID NO: 5 - DNA, 28 bp, Artificial Sequence
  staple sequence
aggcgctgaacggacttcatagaagcgc

SEQ ID NO: 6 - DNA, 40 bp, ARTIFICIAL SEQUENCE
  staple sequence
tataacgtgctttaggtgtctgtagaaccagcaataaaag

SEQ ID NO: 7 - DNA, 48 bp, ARTIFICIAL SEQUENCE
  staple sequence
ctgcatgaatatcagcaccaacagattagcggcgttgacatatcaaaa

SEQ ID NO: 8 - DNA, 38 bp, ARTIFICIAL SEQUENCE
  staple sequence
acggcgctttaccaaatacctaaatagttgttatggtc

SEQ ID NO: 9 - DNA, 32 bp, ARTIFICIAL SEQUENCE
  staple sequence
ttaaactcaaaattttacattaaaatcatggt

SEQ ID NO: 10 - DNA, 46 bp, ARTIFICIAL SEQUENCE
  staple sequence
aatatgacggtaaagaaccagtagtgtcatagccagatgcccgtca

SEQ ID NO: 11 - DNA, 32 bp, ARTIFICIAL SEQUENCE
  staple sequence
ataacgataccactgaccctaatcaccatggt

SEQ ID NO: 12 - DNA, 46 bp, ARTIFICIAL SEQUENCE
  staple sequence
agaaaaaccggtcggaccaccattaccatccaaaggataaacccac

SEQ ID NO: 13 - DNA, 40 bp, ARTIFICIAL SEQUENCE
  staple sequence
tctgaatggggtcggcatcaaaagtaatcacgttcttggt

SEQ ID NO: 14 - DNA, 28 bp, ARTIFICIAL SEQUENCE
  staple sequence
accaaaaaccatttttgaatctctcaga

SEQ ID NO: 15 - DNA, 40 bp, ARTIFICIAL SEQUENCE
  staple sequence
cagcaagacaatatcacgaaaatataatcggtaaccaacc

SEQ ID NO: 16 - DNA, 38 bp, ARTIFICIAL SEQUENCE
  staple sequence
cagtatgcaaattagcacaggcaaaaaatttacaatga

SEQ ID NO: 17 - DNA, 36 bp, ARTIFICIAL SEQUENCE
  staple sequence
catcacacagtccttgacggtcgttctctaatccgc

SEQ ID NO: 18 - DNA, 32 bp, ARTIFICIAL SEQUENCE
  staple sequence
ctgtcgcacctccagccggcaaaagtggtcta

SEQ ID NO: 19 - DNA, 50 bp, ARTIFICIAL SEQUENCE
  staple sequence
actcagaagagaaataagcgaaccaaataagcagcttgcagacccataat

SEQ ID NO: 20 - DNA, 32 bp, ARTIFICIAL SEQUENCE
  staple sequence
agcagccttatggccgtcaaggagcacatcgg

SEQ ID NO: 21 - DNA, 48 bp, ARTIFICIAL SEQUENCE
  staple sequence
gtcaatagtaaagtgcaccgcatgcggccattagctgtacaccctcgg

SEQ ID NO: 22 - DNA, 50 bp, ARTIFICIAL SEQUENCE
  staple sequence
tacccttgaaagtgggacgaccaaaaagtctcaggaggaagcggagcagt

SEQ ID NO: 23 - DNA, 36 bp, ARTIFICIAL SEQUENCE
  staple sequence
tgcggtgaaaaagcgtcctgagcgcgcataaacgta

SEQ ID NO: 24 - DNA, 40 bp, ARTIFICIAL SEQUENCE
  staple sequence
cacacaaactgtaggaagtgtccggtggtagaagtcgtca

SEQ ID NO: 25 - DNA, 32 bp, ARTIFICIAL SEQUENCE
  staple sequence
gataggtcgtagtaatggatacgcgcgccgcc

SEQ ID NO: 26 - DNA, 46 bp, ARTIFICIAL SEQUENCE
  staple sequence
gatagcggcgactggcagtcggcgtgactgtaaccataaggcgaac

SEQ ID NO: 27 - DNA, 40 bp, ARTIFICIAL SEQUENCE
  staple sequence
tctaaaccgaacgtgccaagcatagacagaatagcttctc

SEQ ID NO: 28 - DNA, 32 bp, ARTIFICIAL SEQUENCE
  staple sequence
tggtaagttggattaagcaccccagcctcaca

SEQ ID NO: 29 - DNA, 38 bp, ARTIFICIAL SEQUENCE
  staple sequence
tttggcgagaaagctcttagggtcaacgctacaatact

SEQ ID NO: 30 - DNA, 28 bp, ARTIFICIAL SEQUENCE
  staple sequence
tgttaatttgagcagactcattctagct

SEQ ID NO: 31 - DNA, 48 bp, ARTIFICIAL SEQUENCE
  staple sequence
ccaaatgttgatttcttacctattcagcatcggactcagagactcata

SEQ ID NO: 32 - DNA, 48 bp, ARTIFICIAL SEQUENCE
  staple sequence
gttagcctctgaaacaaatgcttatggtatcagggttaatgtggcatt

SEQ ID NO: 33 - DNA, 46 bp, Artificial Sequence
  staple sequence
gtcaaatgagcctgacaagagaatctatcgaaatcatcttcgtgtt

SEQ ID NO: 34 - DNA, 32 bp, ARTIFICIAL SEQUENCE
  staple sequence
tttcttctgcgtcagtaagaaccagggccttt

SEQ ID NO: 35 - DNA, 38 bp, ARTIFICIAL SEQUENCE
  staple sequence
ggaaaccataacgagctggaaacgtacggatttaaaat

SEQ ID NO: 36 - DNA, 32 bp, ARTIFICIAL SEQUENCE
  staple sequence
acggcgtcaccaatctgccgaagcttgccttt

SEQ ID NO: 37 - DNA, 40 bp, ARTIFICIAL SEQUENCE
  staple sequence
cgctctttgttcagtaacttgactttgagatggcagcaac

SEQ ID NO: 38 - DNA, 50 bp, ARTIFICIAL SEQUENCE
  staple sequence
agaggtttatgggatccaaagcggtcatcatcttgattaagctcattagg

SEQ ID NO: 39 - DNA, 40 bp, ARTIFICIAL SEQUENCE
  staple sequence
aacaccatcttaatccactgttcaatgtctacgagaaaga

SEQ ID NO: 40 - DNA, 28 bp, ARTIFICIAL SEQUENCE
  staple sequence
cggcaacataccaaaggcggctttacgt

SEQ ID NO: 41 - DNA, 36 bp, ARTIFICIAL SEQUENCE
  staple sequence
actcagcggtcagtagcaatgttgaccacctgcaat

SEQ ID NO: 42 - DNA, 44 bp, ARTIFICIAL SEQUENCE
  staple sequence
cagcaaggtagcgacagttgttccttgaacggcgtcgcgtctgc

SEQ ID NO: 43 - DNA, 26 bp, ARTIFICIAL SEQUENCE
  staple sequence
agcaccaaacttgttaaatcccagcg

SEQ ID NO: 44 - DNA, 44 bp, ARTIFICIAL SEQUENCE
  staple sequence
gtactgaatacaaaactcggtatagataagcaggagaaacaagc

SEQ ID NO: 45 - DNA, 32 bp, ARTIFICIAL SEQUENCE
  staple sequence
caacaggcgcgaatatctttttggattcttta

SEQ ID NO: 46 - DNA, 20 bp, ARTIFICIAL SEQUENCE
  staple sequence
gcgcgagcgccaaaatggta

SEQ ID NO: 47 - DNA, 44 bp, ARTIFICIAL SEQUENCE
  staple sequence
tctttaatccagcaatcacctcaccagatacaaactcatcatta

SEQ ID NO: 48 - DNA, 44 bp, ARTIFICIAL SEQUENCE
  staple sequence
gaacatgggcatattatcataaaacgcccacgcaaaaagcggct

SEQ ID NO: 49 - DNA, 47 bp, ARTIFICIAL SEQUENCE
  staple sequence
gaagatttcacgcggcggcaagttgccatctctttaaagaaggtcat

SEQ ID NO: 50 - DNA, 32 bp, ARTIFICIAL SEQUENCE
  staple sequence
gtacggggtgaatcgcaatcttttttaagtgg

SEQ ID NO: 51 - DNA, 26 bp, ARTIFICIAL SEQUENCE
  staple sequence
aagtgacgtttgagagattaccgcgc

SEQ ID NO: 52 - DNA, 25 bp, ARTIFICIAL SEQUENCE
  staple sequence
acgaagtagcggtaaagtagcctct

SEQ ID NO: 53 - DNA, 40 bp, ARTIFICIAL SEQUENCE
  staple sequence
cttcgtcgcagtcgaacaagcggcgccatctgttgacagg

SEQ ID NO: 54 - DNA, 32 bp, ARTIFICIAL SEQUENCE
  staple sequence
tttatagggtttgaattcatgcggagtcaaag

SEQ ID NO: 55 - DNA, 44 bp, ARTIFICIAL SEQUENCE
  staple sequence
agtcatggcgacctggagtaacagaagtacaggtgccataaaca

SEQ ID NO: 56 - DNA, 20 bp, ARTIFICIAL SEQUENCE
  staple sequence
acacgaaggcggctcagcgg

SEQ ID NO: 57 - DNA, 25 bp, ARTIFICIAL SEQUENCE
  staple sequence
tccgacaggagcaggaaacggagta

SEQ ID NO: 58 - DNA, 40 bp, ARTIFICIAL SEQUENCE
  staple sequence
actttcagcgaaggcatttagtcatgataaggacgtgaaa

SEQ ID NO: 59 - DNA, 40 bp, ARTIFICIAL SEQUENCE
  staple sequence
ttcatgactttttttagccatactcatccacaaccagtag

SEQ ID NO: 60 - DNA, 44 bp, ARTIFICIAL SEQUENCE
  staple sequence
cgggatgaaatgttttttccatgaccttctgcacgtaatttaga

SEQ ID NO: 61 - DNA, 26 bp, ARTIFICIAL SEQUENCE
  staple sequence
gcgacgtgtagccacgtattcgccag

SEQ ID NO: 62 - DNA, 32 bp, ARTIFICIAL SEQUENCE
  staple sequence
ggttcaatcatttttatcgaaaggtcgctcat

SEQ ID NO: 63 - DNA, 25 bp, ARTIFICIAL SEQUENCE
  staple sequence
acgctcgcaagagtaaacttggtca

SEQ ID NO: 64 - DNA, 25 bp, ARTIFICIAL SEQUENCE
  staple sequence
aattacatagaaaccaaccacttcg

SEQ ID NO: 65 - DNA, 44 bp, ARTIFICIAL SEQUENCE
  staple sequence
gacccatcaacagggacataaaaagtaaataaacgtaagaaacg

SEQ ID NO: 66 - DNA, 47 bp, ARTIFICIAL SEQUENCE
  staple sequence
acgcaaccggacgctcgacgccattaatacataataacatcactata

SEQ ID NO: 67 - DNA, 32 bp, ARTIFICIAL SEQUENCE
  staple sequence
cctcaacgacttctgccatcagaatgagacag

SEQ ID NO: 68 - DNA, 44 bp, ARTIFICIAL SEQUENCE
  staple sequence
cgaggtcagaaatccaacgcgtcagtttaagccactcagcgtac

SEQ ID NO: 69 - DNA, 26 bp, ARTIFICIAL SEQUENCE
  staple sequence
tgagtataataaatcataggctgaat

SEQ ID NO: 70 - DNA, 47 bp, ARTIFICIAL SEQUENCE
  staple sequence
acccgattctgaacagcttcttgggaagtccatatcatatctggggt

SEQ ID NO: 71 - DNA, 40 bp, ARTIFICIAL SEQUENCE
  staple sequence
cattagcaatgaaaactcaaaagtgttacagcgacgacaa

SEQ ID NO: 72 - DNA, 20 bp, ARTIFICIAL SEQUENCE
  staple sequence
ccctttgtagcagatttcat

SEQ ID NO: 73 - DNA, 20 bp, ARTIFICIAL SEQUENCE
  staple sequence
tcgtcaatctcaaattcgta

SEQ ID NO: 74 - DNA, 32 bp, ARTIFICIAL SEQUENCE
  staple sequence
ggcgctgcgtaaccgtcttcagtgtcaaatca

SEQ ID NO: 75 - DNA, 47 bp, ARTIFICIAL SEQUENCE
  staple sequence
acgttccaagagctcagaagcaataccgaacctgatctcagtaaatc

SEQ ID NO: 76 - DNA, 32 bp, ARTIFICIAL SEQUENCE
  staple sequence
catatttaacctgactattcttgaattaaaaa

SEQ ID NO: 77 - DNA, 32 bp, ARTIFICIAL SEQUENCE
  staple sequence
acgcccctgcaattaaaattaggccacgagga

SEQ ID NO: 78 - DNA, 24 bp, ARTIFICIAL SEQUENCE
  staple sequence
acccttcctgaatgaatgggatac

SEQ ID NO: 79 - DNA, 24 bp, ARTIFICIAL SEQUENCE
  staple sequence
acgcttgtgccaattcatccacga

SEQ ID NO: 80 - DNA, 22 bp, ARTIFICIAL SEQUENCE
  staple sequence
cagtcagccatataactgacca

SEQ ID NO: 81 - DNA, 36 bp, ARTIFICIAL SEQUENCE
  staple sequence
agtggaggttgcattcaaacgatacgtcagccaacg

SEQ ID NO: 82 - DNA, 22 bp, ARTIFICIAL SEQUENCE
  staple sequence
agcaatagaccaaaccatcaat

SEQ ID NO: 83 - DNA, 22 bp, ARTIFICIAL SEQUENCE
  staple sequence
ttcgcatagtgccatgctacac

SEQ ID NO: 84 - DNA, 34 bp, ARTIFICIAL SEQUENCE
  staple sequence
gtcgcaaatgaaccttcaacggcagaagcttaat

SEQ ID NO: 85 - DNA, 34 bp, ARTIFICIAL SEQUENCE
  staple sequence
aacgcagacgacaccattcgggagggtaaagaag

SEQ ID NO: 86 - DNA, 36 bp, ARTIFICIAL SEQUENCE
  staple sequence
aacaagcagaattttcaaagtaagcgttagttgatg

SEQ ID NO: 87 - DNA, 44 bp, ARTIFICIAL SEQUENCE
  staple sequence
aagttgggcatacatacaacgccctgcatacgaaaagacacgtc

SEQ ID NO: 88 - DNA, 34 bp, ARTIFICIAL SEQUENCE
  staple sequence
tttcttgttgacagtcccaagctatttaattgcg

SEQ ID NO: 89 - DNA, 36 bp, ARTIFICIAL SEQUENCE
  staple sequence
caaaaattctaagcagtggcgagattatcagaaaaa

SEQ ID NO: 90 - DNA, 44 bp, ARTIFICIAL SEQUENCE
  staple sequence
ttcttgcacagcaatcaatcaccagaacggaaaacatcctggaa

SEQ ID NO: 91 - DNA, 34 bp, ARTIFICIAL SEQUENCE
  staple sequence
cctagagatgtatgacgcgcatgacaagttgtca

SEQ ID NO: 92 - DNA, 36 bp, ARTIFICIAL SEQUENCE
  staple sequence
ccaacgaagaagcagcattaaccgtcaatgtatcca

SEQ ID NO: 93 - DNA, 36 bp, ARTIFICIAL SEQUENCE
  staple sequence
ctttgcattgggtgaatcattagccttgtactcagg

SEQ ID NO: 94 - DNA, 36 bp, ARTIFICIAL SEQUENCE
  staple sequence
agtctctcctcactaccatgaacaaaatgtaatcca

SEQ ID NO: 95 - DNA, 24 bp, ARTIFICIAL SEQUENCE
  staple sequence
attttctctctttttgcgttcgta

SEQ ID NO: 96 - DNA, 36 bp, ARTIFICIAL SEQUENCE
  staple sequence
ataagacgcatctcgaacgcaatgagtagagtcaat

SEQ ID NO: 97 - DNA, 44 bp, ARTIFICIAL SEQUENCE
  staple sequence
taactttttccgtggaagcattttcatcccgaagttgcggtttg

SEQ ID NO: 98 - DNA, 44 bp, ARTIFICIAL SEQUENCE
  staple sequence
tgcggacgacgtcagtcgcaaggtaaacgcgaacaattcaacga

SEQ ID NO: 99 - DNA, 24 bp, ARTIFICIAL SEQUENCE
  staple sequence
gttggaacgttttttacctttttg

SEQ ID NO: 100 - DNA, 22 bp, ARTIFICIAL SEQUENCE
  staple sequence
ataacgcgagggtatcctagca

SEQ ID NO: 101 - DNA, 36 bp, ARTIFICIAL SEQUENCE
  staple sequence
aacagacgatgattaacagtcgggagagtgccaaga

SEQ ID NO: 102 - DNA, 25 bp, ARTIFICIAL SEQUENCE
  staple sequence
ccgcttcggcgttataacctcacac

SEQ ID NO: 103 - DNA, 25 bp, ARTIFICIAL SEQUENCE
  staple sequence
catggaagcgataaaactctgcagg

SEQ ID NO: 104 - DNA, 25 bp, ARTIFICIAL SEQUENCE
  staple sequence
tcttgaacactcatccttaatacct

SEQ ID NO: 105 - DNA, 25 bp, ARTIFICIAL SEQUENCE
  staple sequence
ctgctttatcaagataatttttcga

SEQ ID NO: 106 - DNA, 25 bp, ARTIFICIAL SEQUENCE
  staple sequence
ttaagagggcgttcagcagccagct

SEQ ID NO: 107 - DNA, 25 bp, ARTIFICIAL SEQUENCE
  staple sequence
tagacataatttatcctcaagtaag

SEQ ID NO: 108 - DNA, 25 bp, ARTIFICIAL SEQUENCE
  staple sequence
gtggtcggcagattgcgataaacgg

SEQ ID NO: 109 - DNA, 25 bp, Artificial Sequence
  staple sequence
ccagcaaggaagccaagatgggaaa

SEQ ID NO: 110 - DNA, 40 bp, ARTIFICIAL SEQUENCE
  staple sequence
ttatcagatctgtaaaacaggtgcgatgtagcaattaaaa

SEQ ID NO: 111 - DNA, 40 bp, ARTIFICIAL SEQUENCE
  staple sequence
catcagggtcctttaccagctttaacgtacctattcagcg

SEQ ID NO: 112 - DNA, 40 bp, ARTIFICIAL SEQUENCE
  staple sequence
tctctttagccagcaatatcggtacatacaaaccagaaaa

SEQ ID NO: 113 - DNA, 40 bp, ARTIFICIAL SEQUENCE
  staple sequence
tggtatcagttgttatagatattcacggcgcttgttcacc

SEQ ID NO: 114 - DNA, 32 bp, ARTIFICIAL SEQUENCE
  staple sequence
ataaatcatattgttatcggtagctcacccat

SEQ ID NO: 115 - DNA, 33 bp, ARTIFICIAL SEQUENCE
  staple sequence
ataacttaacctcaaagtctgaaaccaccaaac

SEQ ID NO: 116 - DNA, 32 bp, ARTIFICIAL SEQUENCE
  staple sequence
gcctacagttaggaacattagagcgacggcct

SEQ ID NO: 117 - DNA, 32 bp, ARTIFICIAL SEQUENCE
  staple sequence
aacagtcggggttaatcgtgccaagattttat

SEQ ID NO: 118 - DNA, 33 bp, ARTIFICIAL SEQUENCE
  staple sequence
cagaggagtggagatttaataccaggcacatca

SEQ ID NO: 119 - DNA, 32 bp, ARTIFICIAL SEQUENCE
  staple sequence
ccttgaatgccaccggaggcggctgtagtgtt

SEQ ID NO: 120 - DNA, 32 bp, ARTIFICIAL SEQUENCE
  staple sequence
ctccaaacaatttagacatggcgcgtgccatg

SEQ ID NO: 121 - DNA, 32 bp, ARTIFICIAL SEQUENCE
  staple sequence
ctcaggaagtcgcagtaggcggaagtactgaa

SEQ ID NO: 122 - DNA, 33 bp, ARTIFICIAL SEQUENCE
  staple sequence
catgaaacgcaaaggtcaatataacttgaccgc

SEQ ID NO: 123 - DNA, 33 bp, ARTIFICIAL SEQUENCE
  staple sequence
gcgcacctaagaaaaagagtaaacaccagcaag

SEQ ID NO: 124 - DNA, 32 bp, ARTIFICIAL SEQUENCE
  staple sequence
agcagaagcaataccgccagcaatgattaaac

SEQ ID NO: 125 - DNA, 32 bp, ARTIFICIAL SEQUENCE
  staple sequence
tcctaagcaaaaaagtttgaattagaaccagc

SEQ ID NO: 126 - DNA, 48 bp, ARTIFICIAL SEQUENCE
  staple sequence
tcaaaatacatctgaaaccattacacaacctgtcatcttcacactact

SEQ ID NO: 127 - DNA, 40 bp, ARTIFICIAL SEQUENCE
  staple sequence
aaaatttagggtacagtcctcatatcatcttcacactact

SEQ ID NO: 128 - DNA, 60 bp, ARTIFICIAL SEQUENCE
  staple sequence
gatgtatccggcatcacaacagaagtcgggagggtagtcgataatcatcttcacactact

SEQ ID NO: 129 - DNA, 44 bp, ARTIFICIAL SEQUENCE
  staple sequence
ataataacgttgaagaagctcagcattatcatcttcacactact

SEQ ID NO: 130 - DNA, 56 bp, ARTIFICIAL SEQUENCE
  staple sequence
acattgacggtaggacgtcaatagtcactctctttaaaagtcatcttcacactact

SEQ ID NO: 131 - DNA, 40 bp, ARTIFICIAL SEQUENCE
  staple sequence
ggcaaccaaaccatgaaaccgtagtcatcttcacactact

SEQ ID NO: 132 - DNA, 48 bp, ARTIFICIAL SEQUENCE
  staple sequence
cagaagtggcggcagaaccaaatccaagataatcatcttcacactact

SEQ ID NO: 133 - DNA, 40 bp, ARTIFICIAL SEQUENCE
  staple sequence
agtttgagcacgtccatctcaccatcatcttcacactact

SEQ ID NO: 134 - DNA, 60 bp, ARTIFICIAL SEQUENCE
  staple sequence
ctttatcactcccaagtgcagcaggaccagcaaggaagccataatcatcttcacactact

SEQ ID NO: 135 - DNA, 44 bp, ARTIFICIAL SEQUENCE
  staple sequence
ccttccagcctgcatagcacaagcaacttcatcttcacactact

SEQ ID NO: 136 - DNA, 56 bp, ARTIFICIAL SEQUENCE
  staple sequence
gacggaaggagtgagagcgccaacggcgcatgaactgtaatcatcttcacactact

SEQ ID NO: 137 - DNA, 40 bp, ARTIFICIAL SEQUENCE
  staple sequence
atctgtctacagtagagtcacatatcatcttcacactact

SEQ ID NO: 138 - DNA, 40 bp, ARTIFICIAL SEQUENCE
  staple sequence
ggtagaagtcgtgttttaccttcttcatcttcacactact

SEQ ID NO: 139 - DNA, 48 bp, ARTIFICIAL SEQUENCE
  staple sequence
cggtacggcaacggaatcttgattgcggagcatcatcttcacactact

SEQ ID NO: 140 - DNA, 60 bp, ARTIFICIAL SEQUENCE
  staple sequence
gatggcagcatttggcaggaggaacagtatgcaaattagctaattcatcttcacactact

SEQ ID NO: 141 - DNA, 44 bp, ARTIFICIAL SEQUENCE
  staple sequence
tatatcaggcatataaccctaagctcattcatcttcacactact

SEQ ID NO: 142 - DNA, 56 bp, ARTIFICIAL SEQUENCE
  staple sequence
cacgtccaaatggtcagcgtcataagagggtctgtttcaatcatcttcacactact

SEQ ID NO: 143 - DNA, 40 bp, ARTIFICIAL SEQUENCE
  staple sequence
tggtatagccagatgcccagcagttcatcttcacactact

SEQ ID NO: 144 - DNA, 48 bp, ARTIFICIAL SEQUENCE
  staple sequence
cacgcggcttaaacttgaacggaagcgcataatcatcttcacactact

SEQ ID NO: 145 - DNA, 40 bp, ARTIFICIAL SEQUENCE
  staple sequence
tgggaagccttcaatttaacatcatcatcttcacactact

SEQ ID NO: 146 - DNA, 60 bp, ARTIFICIAL SEQUENCE
  staple sequence
cagcaatcaagaaggtatacgaagggcgaataagtacgcgtggttcatcttcacactact

SEQ ID NO: 147 - DNA, 44 bp, ARTIFICIAL SEQUENCE
  staple sequence
gtcaggcaagttagtcaaagaacatccttcatcttcacactact

SEQ ID NO: 148 - DNA, 56 bp, ARTIFICIAL SEQUENCE
  staple sequence
catactgactatataaacggtcacattaacccctcacacttcatcttcacactact

SEQ ID NO: 149 - DNA, 40 bp, ARTIFICIAL SEQUENCE
  staple sequence
tggtcttttatcacgaagtctaactcatcttcacactact

SEQ ID NO: 150 - DNA, 44 bp, ARTIFICIAL SEQUENCE
  staple sequence
cctacgagtcagtccttccaaagggaaccgaaccaaacaggcaa

SEQ ID NO: 151 - DNA, 51 bp, ARTIFICIAL SEQUENCE
  staple sequence
cctacgagtcagtcctttagggatggaggtggcgtaacagaagtgcgagaa

SEQ ID NO: 152 - DNA, 40 bp, ARTIFICIAL SEQUENCE
  staple sequence
cctacgagtcagtcctcggtataacctgtcaagaagacaa

SEQ ID NO: 153 - DNA, 48 bp, ARTIFICIAL SEQUENCE
  staple sequence
cctacgagtcagtcctggcatttaaacataaaacggggaataataacc

SEQ ID NO: 154 - DNA, 44 bp, ARTIFICIAL SEQUENCE
  staple sequence
cctacgagtcagtcctaaatggtaaagatgggacgctcggcgcc

SEQ ID NO: 155 - DNA, 51 bp, ARTIFICIAL SEQUENCE
  staple sequence
cctacgagtcagtcctaaatcagtaacaggcataaacaaaactatgaatgg

SEQ ID NO: 156 - DNA, 40 bp, ARTIFICIAL SEQUENCE
  staple sequence
cctacgagtcagtcctaaaataatccacttaaaataccat

SEQ ID NO: 157 - DNA, 48 bp, ARTIFICIAL SEQUENCE
  staple sequence
cctacgagtcagtcctatgagggaatagcaaggaaagacgcgccagcg

SEQ ID NO: 158 - DNA, 44 bp, ARTIFICIAL SEQUENCE
  staple sequence
cctacgagtcagtcctgcatgaagataagcaggtcaatagatgt

SEQ ID NO: 159 - DNA, 51 bp, ARTIFICIAL SEQUENCE
  staple sequence
cctacgagtcagtcctcatcgaacagaacggcaacaaatgcttaaaagcgg

SEQ ID NO: 160 - DNA, 40 bp, ARTIFICIAL SEQUENCE
  staple sequence
cctacgagtcagtccttccagaacacgaacagggtctgtt

SEQ ID NO: 161 - DNA, 44 bp, ARTIFICIAL SEQUENCE
  staple sequence
cctacgagtcagtcctggaaacacttcttgcattcctgaatgaa

SEQ ID NO: 162 - DNA, 51 bp, ARTIFICIAL SEQUENCE
  staple sequence
cctacgagtcagtcctcaatcaccttcgccgcgctttagcgttacgaacaa

SEQ ID NO: 163 - DNA, 40 bp, ARTIFICIAL SEQUENCE
  staple sequence
cctacgagtcagtcctctcagcggcaaatttaggtgcttg

SEQ ID NO: 164 - DNA, 48 bp, ARTIFICIAL SEQUENCE
  staple sequence
cctacgagtcagtcctcgtttggtagattagaggacggttaagaaata

SEQ ID NO: 165 - DNA, 48 bp, Artificial Sequence
  staple sequence
cctacgagtcagtcctcggcgttaatgattgaagattgcgtccactgc

SEQ ID NO: 166 - DNA, 33 bp, artificial
  staple sequence
  misc_structure (1)..(1): biotin moiety
tcgtttggtagattagaggacggttaagaaata

SEQ ID NO: 167 - DNA, 32 bp, Artificial Sequence
  staple sequence
  misc_structure (1)..(1): biotin moiety
cggcgttaatgattgaagattgcgtccactgc

SEQ ID NO: 168 - DNA, 15 bp, artificial
  staple sequence
  misc_structure (15)..(15): cholesterol tag
ggactgactcgtagg

SEQ ID NO: 169 - DNA, 15 bp, artificial sequence
  staple sequence
  misc_structure (1)..(1): cholesterol tag
agtagtgtgaagatg

SEQ ID NO: 170 - DNA, 36 bp, Artificial Sequence; identical to SEQ ID NO: 17
  analyte strand

(19) United States
(12) Patent Application Publication
(10) Pub. No.: US 2024/0280530 A1
(43) Pub. Date: Aug. 22, 2024

(54) NANOMECHANICALLY ACTUATED NUCLEIC ACID NANOPORE

(71) Applicant: UCL BUSINESS LTD, London (GB)

(72) Inventors: Stefan HOWORKA, London (GB); Yongzheng XING, London (GB)

(21) Appl. No.: 18/570,995

(22) PCT Filed: Jun. 17, 2022
(86) PCT No.: PCT/EP2022/066627

§ 371 Date: Dec. 15, 2023

(30) Foreign Application Priority Data
Jun. 18, 2021 (GB) 2108821.6

(51) Int. Cl. G01N 27/327

(52) U.S. Cl. CPC G01N 27/3278

(57) ABSTRACT

A membrane-spanning actuatable nucleic acid nanopore is provided. The nanopore comprises: one or more polynucleotide strands that provide a scaffold component and a plurality of polynucleotide strands that provide a plurality of staple components wherein each of the plurality of staple components hybridise to the scaffold component; and a trigger that is actuatable in response to a stimulus. Actuation of the trigger results in a detectable conformational change of the nanopore from a first conformation to at least a second conformation. Detection may be via a signal change, such as via fluorescence (e.g. FRET) or a change in an electrical signal readout. Semi-fluid membranes, and sensor devices comprising the actuatable nucleic acid nanopores are also provided.

### FIELD OF THE INVENTION

The present invention relates to novel membrane nanostructures and their uses. In particular it relates to wide-channel membrane nucleic acid nanopores in the applications of protein sensing and molecular gate creation.

### BACKGROUND OF THE INVENTION

Nanopores are membrane spanning polymers and complexes that can define a perforation and thereby form a channel in semi-fluid lipid bilayer or polymer that forms a partition between two fluids, typically liquids, suitably aqueous solutions, through which ions and certain molecules may pass. Membrane spanning nanopores composed of nucleic acid duplexes, in particular DNA duplexes, represent a possibility for developing sensing nanopores.

DNA nanostructures have been obtained from a structural core of six hexagonally arranged, interlinked DNA duplexes that create a hollow channel (see, for example, Douglas S. M., Marblestone A. H., Teerapittayanon S., Vazquez A., Church G. M., Shih W. M. Nucleic Acids Res. 37, 5001-5006 (2009); Zheng J., et al. Nature 461, 74-77 (2009); Rothemund P. W. Nature 440, 297-302 (2006); Fu J., et al. Nat. Nanotechnol. 9, 531-536 (2014); Burns J. R., et al. Angew. Chem. Int. Ed. 52, 12069-12072 (2013); Seifert A., Göpfrich K., Burns J. R., Fertig N., Keyser U. F., Howorka S. ACS Nano 9, 1117-1126 (2015); Langecker et al. (2012) Science, Vol. 338, Issue 6109, pp. 932-936; and Burns et al. (2013) Nano Lett., 13, 6, 2351-2356). Membrane insertion was achieved through equipping the structures' exterior with hydrophobic lipid anchors.

Modular construction principles for DNA nanopore design has enabled customized pore diameter when used with nanostructures that insert into solid state substrates (Göpfrich et al, Nano. Lett., 15(5), 3134-3138 (2015); WO 2013/083983) and installation of a controllable gate to regulate transport but without initiating any conformational or dimensional change in the nanopore itself (Burns J. R., Seifert A., Fertig N., Howorka S. A., Nat. Nanotechnol. 11, 152-156 (2016)). Circular nanotubes synthesized from DNA are also known in the art (Zheng et al. J. Am. Chem. Soc., 136, 10194-10197 (2014)), whilst nanocarriers, as opposed to membrane-spanning nanopores, intended for targeted drug delivery that undergo opening after exposure to an aptamer are also known (Hamid et al. (2019) Nature Reviews Genetics, 21, 5-26).

To be useful as a sensor for a range of biomolecular analytes such as circulating antibodies, cancer or pathogen biomarkers, suitable membrane channels formed by a nucleic acid nanopore are typically required to meet certain criteria, namely:
1) the channel lumen should be at least about 3 nm wide to accommodate the large biomolecule molecules inside or close to the opening of the channel lumen; binding of the large biomolecule close to or within the channel results in higher read-out sensitivity than when analytes bind in the general vicinity of the pore entrance;
2 the pores should be structurally defined and resistant to flex or conformational distortion to attain a constant base level in the electrical read-out (i.e. reduce background noise); and
3) the pore dimensions should be easily tunable to adapt them to different biomolecule sizes—e.g. they should be configured to be conformationally or dimensionally appropriate for an analyte or biomolecule that may interact with or pass through the pore.

Membrane spanning nucleic acid structurally defined nanopores with a minimum internal pore width of several nanometers are described in WO-2018/011603-A1, and also in WO2020/025974-A1.

In a conventional nanopore sensor the detection of an analyte occurs through the occlusion of the pore leading to a change in a detectable signal. The signal may be the result of a measurement system created by placing the nanopore within an insulating semi-fluid membrane and measuring voltage-driven ion flow through the nanopore in the presence of a soluble analyte. A change in signal may be detected as a reduction in a measurable electrical current passing through the pore. Such an obstruction of current flow through the pore is termed current blockade. A change in the flow of ionic species through the pore may be measured as a change in electrical current, electrical potential (e.g. voltage) or impedance. Further information about the analyte may be revealed by distinctive ion current signatures, such as the duration and extent of current block and the variance of current levels. Clearly, the highest degree of detection occurs when the current blockade is maximal, however, this often requires the size of the pore lumen to be engineered to correspond closely to the expected size of the analyte that is to be detected. Consequently, detection of small analytes or analytes that may vary in size, such as small molecules or fragments of larger analyte molecules such as oligopeptides, can be difficult to detect as the signal amplitude may be low or variable if near complete current blockade is not possible. Hence, the fidelity of signal output is somewhat dependent upon electrical measurements that are subject to inherent variation in the underlying physical or biological system and/or measurement noise that is inevitable due the tiny magnitude of the signals being measured. Hence, it would be desirable to increase the range of detectable output available to a nucleic acid nanopore and, in particular, to reduce the dependence upon the analyte to match the size of the pore in order to achieve a detectable change in output signal, such as an electrical current blockade.

The present invention addresses the deficiencies in the art. These and other uses, features and advantages of the invention should be apparent to those skilled in the art from the teachings provided herein.

### SUMMARY OF THE INVENTION

The present invention relates to the design and implementation of a nanomechanically actuatable nucleic acid nanopore, as well as systems devices and methods that incorporate this nanostructure.

A first aspect of the invention provides a membrane-spanning actuatable nucleic acid nanopore, the nanopore comprising:
one or more polynucleotide strands that provide a scaffold component and a plurality of polynucleotide strands that provide a plurality of staple components wherein each of the plurality of staple components hybridise to the scaffold component; and
a trigger that is actuatable in response to a stimulus;
wherein actuation of the trigger results in a conformational change of the nanopore from a first conformation to at least a second conformation, and wherein the conformational change is detectable.

A second aspect of the invention provides a membrane into which is inserted at least one membrane-spanning actuatable nucleic acid nanopore as described herein.

A third aspect of the invention provides sensor device, wherein the sensor device comprises a membrane as described herein and a fluorescence measurement apparatus.

A fourth aspect of the invention provides a sensor device comprising a membrane-spanning nucleic acid nanopore as defined herein.

A fifth aspect of the invention provides method for sensing the presence of an analyte comprising:
I. providing a sensor device as defined herein;
II. contacting a nanopore comprised within the sensor device with an analyte and establishing a flow of ions through the nanopore or an electron flow across the nanopore; and
III. measuring an electrical signal across the nanopore,
wherein the sensing comprises analyte detection or characterisation, wherein a change in the electrical measurement is indicative of the presence of the analyte.

A sixth aspect of the invention provides a method for sensing the presence of an analyte comprising:
(a) providing a sensor device as defined herein;
(b) contacting a nanopore comprised within the sensor device with an analyte that acts as a stimulus to initiate a conformational change of the nanopore from a first conformation to at least a second conformation; and
(c) measuring an output signal response via a fluorescence resonance energy transfer (FRET) technique.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 is a representation of a nucleic acid nanopore of a type used in an embodiment of the invention (A) shows a planar view of the nanopore with orientation of the main scaffold strands shown as cylinders; (B) shows a reverse view with hydrophobic anchor placement visible—in this case cholesterol shown as cones; (C) the nanopore structure is in the processes of insertion into a lipid membrane; . . . .

FIG. 2 is a schematic representation of an embodiment of the invention that shows a plan view of a square actuatable nucleic acid nanopore transitioning from a first conformation (A) to an second conformation (B) in response to an external stimulus shown as a single stranded nucleic acid (*).

FIG. 3 shows a graph depicting the transition states between first conformation and second conformation in a nanopore of the type shown in FIG. 2. SP0 is first conformation (reduced lumenal cross-sectional area), SP2 is a transitional conformation and SP4 is second conformation (maximal lumenal cross-sectional area), with the proportion of pores in the configuration as a fraction of 1.0 (i.e. 100%) shown on the y axis. The data was derived from the images shown in FIG. 5 below.

FIG. 4 shows is a representation of an embodiment of the invention that shows a plan view of a square actuatable nucleic acid nanopore transitioning between first and second conformations; two different fluorophores of a FRET dye pair, green-Atto565 (G) and red-Atto647 (R), are located at opposing vertices of the nanopore such that the physical distance between the fluorophores changes upon actuation of the nanopore and transition from the first to the second confirmation.

FIG. 5 shows a series of transmission electron microscope (TEM) images of membranes with nanomechanically actuatable nucleic acid nanopores embedded within, (i) shows nanopores in a first SP0 conformational configuration prior to transition in response to an external stimulus; (ii) and (iii) show images with nanopores in the second SP4 conformational configuration after application of a stimulus.

FIG. 6 shows confocal microscopy images of GUV vesicles comprising fluorophore labelled embedded nanomechanically actuatable nucleic acid nanopores of the type shown in FIG. 4. (A) shows green signal and (B) shows red signal in SP4 square nanopores, (C) show lack of FRET in expanded nanopores, (D) shows the vesicles without fluorescence; (E) and (F) show green and red signal respectively for SP0 nanopores, whilst (G) shows FRET signal in the SP0 configuration, (H) shows the vesicles without fluorescence.

FIG. 7 shows (A) SP0 nanomechanical pore and (B) SP2 nanomechanical pore analysis showing an example electrical signal trace (−50 mV), IV curve and conductance histogram (+20 mV).

FIG. 8 shows a planar view representation of an expanded nanomechanical pore according to one embodiment of the invention. The structure at the vertices is highlighted in the expanded box which shows a region of single stranded DNA (SS) and a region of double stranded DNA (DS).

FIG. 9 shows a schematic representation of an embodiment of the invention that shows a plan view of a square actuatable nucleic acid nanopore that is responsive to a protein analyte. (A) shows a pore without protein binding sites (M0) which remains in a first conformation and is not-actuated in response to the presence of the protein stimulus. (B) shows a pore with one protein binding site (M1) in the form of a receptor, the pore transitions from a first conformation to a second conformation upon binding of the protein analyte to the receptor. (C) shows a pore with two protein binding sites (M2) before and after addition of the protein analyte.

FIG. 10 shows (A) M0 nanomechanical pore, (B) M1 nanomechanical pore and (C) M2 nanomechanical pore analyses showing example electrical signal traces (−50 mV), IV curve and conductance histogram (+20 mV). The embodiments shown relate to a situation where the receptor is in the form of a biotin tag applied to the pore in the locations shown in FIG. 9 for M0, M1 and M2, and streptavidin protein in solution as the protein analyte.

### DETAILED DESCRIPTION OF THE INVENTION

Prior to setting forth the invention, a number of definitions are provided that will assist in the understanding of the invention.

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN:1934-3647); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). Each of these general texts is herein incorporated by reference.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘membrane’ as used herein is an enclosing or separating selectively-permeable boundary, partition, barrier or film. The membrane has two sides or surfaces which may be named the cis and trans side respectively. The membrane is thin (i.e. has a thickness substantially less than its width and length) allowing it to be spanned by the nanopore. In the context of the present invention, the membrane thickness is the typically in the nanometre (10-9 metre) range. The arrangement of the membrane is not limited and may assume any form, for example, a liposome, and a vesicle or as a planar or a non-planar sheet. Specific examples of membranes useful in the present invention include lipid bilayers, polymeric films, or solid state substrates.

The term ‘solid state membrane’ or ‘solid state substrate’ as used herein refers to a membrane or partition formed from a solid state substance—i.e. not a semi-fluid membrane—in which one or more apertures are provided. One or more nanopores may be positioned within the respective one or more apertures disclosed for example in U.S. Pat. No. 8,828,211, hereby incorporated by reference. The solid state membrane may comprise either or both of organic and inorganic materials, including, but not limited to, microelectronic materials, whether electrically conducting, electrically semiconducting, or electrically insulating, including materials such as II-IV and III-V materials, oxides and nitrides, such as silicon nitride, Al₂O₃, and SiO₂, Si, MoS₂, solid state organic and inorganic polymers such as polyamide, plastics such as Teflon®, or elastomers such as two-component addition-cure silicone rubber, and glasses. A membrane may be formed from monatomic layers, such as graphene, or layers that are only a few atoms thick such as those disclosed in U.S. Pat. No. 8,698,481, and U.S. Patent Application Publication 2014/174927, both hereby incorporated by reference. More than one layer of material can be included, such as more than one graphene layer, as disclosed in US Patent Application Publication 2013/309776, incorporated herein by reference. Suitable silicon nitride membranes are disclosed in U.S. Pat. No. 6,627,067, and the membrane may be chemically functionalized, such as disclosed in U.S. Patent Application Publication 2011/053284, both hereby incorporated by reference. Such a structure is disclosed for example in U.S. Pat. No. 8,828,211, hereby incorporated by reference. The internal walls of the apertures may be coated with a functionalised coating, such as disclosed in published application WO 2009/020682. The one or more apertures may be hydrophobic or provided with a hydrophobic coating to assist the provision of the one or more nanopores in the respective one or more apertures. Suitable methods for providing apertures in solid state substrates are disclosed in published applications WO 03003446 and WO 2016/187519.

The term ‘modular’ as used herein refers to the use of one or more units, or modules, to design or construct a whole or part of a larger system. In the context of the present invention it refers to the use of individual modules, sub-units or building blocks to construct a nanopore. The modules may be each the same or the modules may be different. To form the nanopore, the individual modules may be connected or inter-linked to one or more other modules. The means of connection between modules may be by chemical or physical means, such as covalent or non-covalent chemical bonding or by electrostatic or other attractive forces. Alternatively, or in addition, the means of connection may be via an additional module, bracing member, portion or linkage. The modular design of a nanopore may comprise a frame or framework of modules, and additional, typically smaller, sub-modules that connect, or support the frame, acting as struts or bracing members. The modules span the membrane to enable formation the channel of the nanopore; the sub-modules do not generally span the membrane and are intended only as structural support in the nanopore. Some modules may sit on a surface of the membrane in order to stabilise membrane insertion of membrane spanning modules. Such surface located modules may adopt a raft-like configuration and serve as location points for one or more anchors. The design of the modules and sub-modules, or how they connect, may be chosen to support and strengthen the formed channel of the nanopore such that it maintains its shape and conformational integrity when inserted in a membrane. The modules or sub-modules may be formed of nucleic acids, typically DNA. Each individual unit may be assembled by DNA origami techniques described elsewhere herein using suitably selected scaffold and staple strands. Each individual unit may be assembled by DNA/RNA origami techniques described elsewhere herein using suitably selected scaffold and staple strands in order to create a higher order structure—e.g., a secondary structure having defined geometric parameters. Such secondary structure may include the formation of A-, B- or Z-form double helices (duplex), triplex, quadruplex, hairpin loops, and trefoil structures as well as combinations of such structures.

The term ‘nucleic acid’ as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, and are typically manufactured synthetically, but may also be isolated from natural sources. Nucleic acids may further include modified DNA or RNA, for example DNA or RNA that has been methylated or that has been subject to chemical modification, for example 5′-capping with 7-methylguanosine, 3′-processing such as cleavage and polyadenylation, and splicing, or labelling with fluorophores or other compounds. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Hence, where the terms ‘DNA’ and ‘RNA’ are used herein it should be understood that these terms are not limited to only include naturally occurring nucleotides. Sizes of nucleic acids, also referred to herein as ‘polynucleotides’ are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 100 nucleotides in length are typically called ‘oligonucleotides’.

As used herein, the terms ‘3’′ (‘3 prime’) and ‘5′’ (‘5 prime’) take their usual meanings in the art, i.e. to distinguish the ends of polynucleotides. A polynucleotide has a 5′ and a 3′ end and polynucleotide sequences are conventionally written in a 5′ to 3′ direction. The term ‘complements of a polynucleotide molecule’ denotes a polynucleotide molecule having a complementary base sequence and reverse orientation as compared to a reference sequence.

The term ‘duplex’ is used herein refers to double-stranded DNA, meaning that the nucleotides of two complimentary DNA sequences have bonded together and then coiled to form a double helix (assuming A-, B- or Z-form), or also single-stranded RNA (ssRNA) that has annealed to a complimentary DNA sequence to generate an RNA-DNA hybrid (RDH) duplex. An RDH nanostructure may comprise a single RNA scaffold sequence with multiple shorter hybridised DNA sequences (e.g. DNA oligonucleotides) acting as staples forming a series of RDH duplexes along the length of the RNA scaffold thereby defining higher order structures.

According to the present invention, homology to the nucleic acid sequences described herein is not limited simply to 100%, 99%, 98%, 97%, 95% or even 90% sequence identity. Many nucleic acid sequences can demonstrate biochemical equivalence to each other despite having apparently low sequence identity. In the present invention homologous nucleic acid sequences are considered to be those that will hybridise to each other under conditions of low stringency (Sambrook J. et al, Molecular Cloning: a Laboratory Manual, Cold Spring Harbor Press, Cold Spring Harbor, NY). However, it may be desired in some cases to distinguish between two sequences which can hybridise to each other but contain some mismatches—an “inexact match”, “imperfect match”, or “inexact complementarity”—and two sequences which can hybridise to each other with no mismatches—an “exact match”, “perfect match”, or “exact complementarity”. Further, possible degrees of mismatch are considered.

As used herein, the term ‘nanostructure’ refers to a predesigned two or three dimensional molecular structure typically comprised from a biopolymer, suitably a naturally or non-naturally occurring nucleic acid, which structure has at least one dimension or an aspect of its geometry that is within the nanoscale (i.e. 10-9 metres). Nanoscale structures suitably have dimensions or geometry of less than around 100 nm, typically less than 50 nm, and most suitably less than 20 nm. Nanoscale structures suitably possess dimensions or geometry greater than around 0.1 nm, typically greater than around 1 nm, and optionally greater than around 2 nm. Assembly of nucleic acid nanostructures may occur spontaneously in solution, such as by heating and cooling a mixture of DNA strands of preselected sequences, or may require presence of additional co-factors including, but not limited to, nucleic acid scaffolds, nucleic acid aptamers, nucleic acid staples, co-enzymes, and molecular chaperones. Where desired nanostructures result from one or more predesigned spontaneously self-folding nucleic acid molecules, such as DNA, this is typically referred to as nucleic acid ‘origami’. Rational design and folding of DNA to create two dimensional or three dimensional nanoscale structures and shapes is known in the art (e.g. Rothemund (2006) Nature 440, 297-302). In the classical scaffold-and-staple approach, one or more long biogenic scaffold strand component(s) is folded into a defined DNA nanostructure with a staple component consisting of shorter synthetic staple oligonucleotides. Classical DNA nanostructures are formed of bundles of parallel aligned DNA duplexes that are arranged into polygons that define a channel and puncture a membrane bilayer, to create a hollow conduit that passes through the membrane from one side to the other. Suitably scaffold structures may be based off M13 or phix174 sequences, to which a plurality of smaller staple and linker sequences are configured to achieve the desired three-dimensional nanostructural geometry. In embodiments of the present invention alternative scaffolds may be utilised and may comprise artificial, or non-naturally occurring, sequences that are designed specifically for the task of nanostructural modular assembly. Typically, such sequences will be non-repetitive and with base selection that is optimised to facilitate nucleic acid hybridisation between component modules under conditions that favour nanostructure assembly.

In embodiments of the invention nanopores having the configurations and staple/scaffold strand sequences as described in the examples that follow may be considered to fall within the scope of the invention. It will be appreciated that the analyte binding sequences of the disclosed nanomechanical nanopores may also be modified to comprise one or more alternative binding moiety, such as a polynucleotide or a polypeptide that is capable of binding to an analyte—see below for further examples.

The nucleic acid sequences that form the nanostructures will typically be manufactured synthetically, although they may also be obtained by conventional recombinant nucleic acid techniques. DNA constructs comprising the required sequences may be comprised within vectors grown within a microbial host organism (such as E. coli). This would allow for large quantities of the DNA to be prepared within a bioreactor and then harvested using conventional techniques. The vectors may be isolated, purified to remove extraneous material, with the desired DNA sequences excised by restriction endonucleases and isolated, such as by using chromatographic or electrophoretic separation.

The term ‘amino acid’ in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=IIe; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A ‘polypeptide’ is a polymer of amino acid residues joined by peptide bonds, whether produced naturally or in vitro by synthetic means. Polypeptides of less than around 12 amino acid residues in length are typically referred to as ‘peptides’ and those between about 12 and about 30 amino acid residues in length may be referred to as ‘oligopeptides’. The term ‘polypeptide’ as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term ‘protein’ is used herein to refer to a macromolecule comprising one or more polypeptide chains.

The term ‘folded protein’ as used herein refers to a protein that has acquired some three-dimensional shape after translation of the polypeptide chain from which it is formed (the primary structure). The term may refer to the secondary structure of the protein which is typically the first stage of the folding process where local three-dimensional structures are formed, for example, alpha helices or beta sheets. The term may more typically refer to the tertiary structure of a protein where the secondary structures of the protein have folded to stabilise the structure through hydrophobic or covalent interactions. The term also encompasses proteins having a quaternary structure where one or more protein subunits are assembled. As appropriate, the folded protein may also be termed the ‘native’ protein structure, and may be the form of the protein that exhibits its biological function.

The term ‘interior width’ when used herein refers to a straight distance spanning the interior of the channel (e.g. the lumen) from a location on an interior face of one wall to an interior face of an opposing wall in a plane perpendicular to the longitudinal axis of the channel (i.e. in cross section). The interior width of the channel may be constant along its longitudinal axis or it may vary due to the presence of one or more constrictions. The ‘minimum interior width’ is a minimum interior width along the longitudinal axis of the channel between an entrance and an exit of the channel. The minimum interior width of a channel defines the maximum size of an object, such as an analyte, that may pass through the channel. In instances where the lumen is comprised of a regular polygonal shape in cross section, the interior width may correspond to the internal diameter of the lumen. The lumen will, therefore, possess a cross sectional area (CSA), which may change as the shape of the nanopore transitions from first to second, or further, conformations. However, it will be appreciated that in some embodiments of the invention the configuration of the nanopore will be such that the nanopore may not have a lumen with a regular polygonal shape in cross section such that there may be several interior widths along its length.

As used herein the term ‘hydrophobic’ refers to a molecule having apolar character including organic molecules and polymers. Examples are saturated or unsaturated hydrocarbons. The molecule may have amphipathic properties.

As used herein, the term ‘hydrophobically-modified’ relates to the modification (joining, bonding or otherwise linking) of a polynucleotide strand with one or more hydrophobic moieties. A ‘hydrophobic moiety’ as defined herein is a hydrophobic organic molecule. The hydrophobic moiety may be any moiety comprising non-polar or low polarity aliphatic, aliphatic-aromatic or aromatic chains. Suitably, the hydrophobic moieties utilised in the present invention encompass molecules such as long chain carbocyclic molecules, polymers, block co-polymers, and lipids. The term ‘lipids’ as defined herein relates to fatty acids and their derivatives (including tri-, di-, monoglycerides, and phospholipids), as well as sterol-containing metabolites such as cholesterol. The hydrophobic moieties comprised within the embodiments of the present invention are capable of forming non-covalent attractive interactions with phospholipid bilayers, such as the lipid-based membranes of cells and act as membrane anchors for the nanopore. According to certain embodiments of the present invention suitable hydrophobic moieties, such as lipid molecules, possessing membrane anchoring properties may include sterols (including cholesterol, derivatives of cholesterol, phytosterol, ergosterol and bile acid), alkylated phenols (including methylated phenols and tocopherols), flavones (including flavanone containing compounds such as 6-hydroxyflavone), saturated and unsaturated fatty acids (including derivatives such as lauric, oleic, linoleic and palmitic acids), and synthetic lipid molecules (including dodecyl-beta-D-glucoside). The anchors for the polymer membrane may be the same as for lipid bilayers or they may be different. The specific hydrophobic moiety anchor may be selected based on the binding performance of the membrane chosen.

The nanopore of the present invention may comprise two or more membrane anchors that act to attach or connect or anchor the hydrophilic DNA nanopore to the generally hydrophobic membrane (lipid bilayer or polymer). The lipid anchors are attached to the pore or comprised within modules that form part of overall the nanostructure of the pore. Suitably attachment is via DNA oligonucleotides that carry the lipid anchor, suitably cholesterol, at the 5′ or 3′ terminus. Polynucleotides or oligonucleotides may be functionalized using a modified phosphoramidite in the strand synthesis reaction, which is easily compatible for the addition of reactive groups, such as cholesterol and lipids, or attachment groups including thiol and biotin. Enzymic modification using a terminal transferase can also be used to incorporate an oligonucleotide, which incorporates a modification such as an anchor, to the 3′ of a single stranded nucleic acid (e.g. ssDNA). These lipid modified anchor strands may hybridize via ‘adaptor’ oligonucleotides to corresponding sections of the DNA sequence forming the scaffold section of the pore. Alternatively, the lipid anchors are assembled with the pore using lipid-modified oligonucleotides that contribute as either the scaffold or staple strands. A combination of approaches to anchoring using two or more membrane anchors may also be adopted wherein anchors are incorporated into one or all of a scaffold strand, a staple strand and an adaptor oligonucleotide. Cholesterol has been found to be a particularly suitable lipid for use as an anchor in the present invention. The use of other lipids as anchors is contemplated, although it may be expected that there is a particular preference for a particular lipid, and a given number of membrane anchors, for a given membrane.

In an alternative embodiment of the invention, the hydrophobic modification is comprised within one or more synthetic nucleic acids (XNAs) incorporated into the nanopore structure itself.

In a specific embodiment of the invention the hydrophobic modification is a lipid, and may be selected from the group consisting of: sterols; alkylated phenols; flavones; saturated and unsaturated fatty acids; and synthetic lipid molecules (including dodecyl-beta-D-glucoside). In further embodiments:
the sterols are selected from the group consisting of: cholesterol; derivatives of cholesterol; phytosterol; ergosterol; and bile acid;
the alkylated phenols are selected from the group consisting of: methylated phenols; dolichols and tocopherols;
the flavones are selected from the group consisting of: flavanone containing compounds; and 6-hydroxyflavone;
the saturated and unsaturated fatty acids are selected from the group consisting of: derivatives of lauric acid; oleic acid; linoleic acid; and palmitic acids;
and/or the synthetic lipid molecule is dodecyl-beta-D-glucoside.

The membrane in which the nanopore of the present invention may be inserted may be of any suitable type. Depending on the intended use, the membrane may be a lipid bilayer or a polymer sheet or film. The membrane is suitably an amphiphilic layer. The amphiphilic layer may be a monolayer or a bilayer. An amphiphilic layer is a layer formed from amphiphilic molecules which have both hydrophilic and lipophilic properties. The amphiphilic molecules may be synthetic or naturally occurring. The lipophilic properties of the molecules comprising the membrane promote anchoring by lipid anchors or other hydrophobic anchoring regions of the nanopore. It is surprising that nucleic acid nanostructures of the invention are able to be inserted successfully into membranes at all given that the hydrophobic nature of the membrane leads to repulsion of the predominantly negatively charged DNA backbone. It might be expected that the nanostructures of the invention would simply form clustered aggregates on one surface of the membrane as is often the case with complex nucleic acid nanostructures that fail to insert successfully into an amphiphilic mono- or bilayer. The ability of the nanostructures to be embedded within such membranes is, therefore, surprising and most unexpected.

In a specific embodiment of the invention the amphiphilic layer may be a lipid bilayer. The lipid composition may comprise naturally-occurring lipids such as phospholipids and bipolar tetraether lipids, and/or artificial lipids. The lipids typically comprise a head group, an interfacial moiety and two hydrophobic tail groups which may be the same or different. Suitable head groups include, but are not limited to, neutral head groups, zwitterionic head groups, negatively charged head groups and positively charged headgroups. The head group or the tail group of the lipids may be chemically-modified.

Proprietary and non-proprietary synthetic polymer films or sheets are widely used in ‘chip-based’ nanopore sequencing and analytical sensor applications such as the MinION® system sold by Oxford Nanopore Technologies®; the GS FLX+® and the GS Junior® System sold by Roche®; the HiSeq®, Genome Analyzer IIx®, MiSeq® and the HiScanSQ® systems sold by Illumina®; the Ion PGM® System and the Ion Proton System® sold by Life Technologies; the CEQ® system sold by Beckman Coulter®; and the PacBio RS® and the SMRT® system sold by Pacific Biosciences®. The ability of nanopores to insert successfully into polymer membranes of this type allows these systems to be adapted for diverse folded protein sensing applications, for example.

Non-naturally occurring amphiphiles and amphiphiles which form an amphiphilic membrane layer are known in the art and include, for example, block copolymers (Gonzalez-Perez et al., Langmuir, 2009, 25, 10447-10450). The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane may be chosen one of the membranes disclosed in PCT/GB2013/052767, hereby incorporated by reference in its entirety. The amphiphilic molecules may be chemically-modified or functionalised to facilitate coupling an insertion of the nanostructure. The membrane can comprise both a lipid and an amphiphilic polymer such as disclosed in PCT/US2016/040665.

Polymer-based membrane may be formed of any suitable material. Typically, synthetic membranes are composed of amphiphilic synthetic block copolymers. Examples of hydrophilic block copolymers are poly(ethylene glycol) (PEG/PEO) or poly(2-methyloxazoline), while examples of hydrophobic blocks are polydimethylsiloxane (PDMS), poly(caprolactone (PCL), poly(lactide) (PLA), or poly(methyl methacrylate) (PMMA). In embodiments, the polymer membrane used may be formed from the amphiphilic block copolymer poly 2-(methacryloyloxy)ethyl phosphorylcholine-b-disisopropylamino) ethyl methacrylate (PMPC-b-PDPA). DNA nanopores may be inserted into the walls of such polymersomes through incubation. Without wishing to be bound by theory, it is believed that one process of insertion broadly involves first steps of membrane tethering, followed by second steps of orientation of the DNA pore relative to the membrane to achieve complete insertion. This however requires lipid membrane anchors to be comprised within, or at least attached to, the pores, without which insertion does not take place. For alternative configurations of the invention, particularly where nucleic acid analogues (e.g. XNAs) comprise some or all of the backbone of the scaffold and/or staple strands, it is envisaged that more complex insertional dynamics may be observed. It will be appreciated that where the scaffold and staple strands incorporate synthetic biopolymer backbone constituents capable of mediating a hydrophobic interaction with a membrane, the insertion process may or may not involve initial stages of coplanar alignment with the membrane followed by a phase of insertion.

The membrane is typically planar, although in certain embodiments it may be curved or shaped. Amphiphilic membrane layers may also be supported. Suitably the membrane is a lipid bilayer or monolayer. Methods for forming lipid bilayers are known in the art such as disclosed in International Application Number PCT/GB2008/000563. Lipid bilayers are commonly formed by the method of Montal and Mueller (Proc. Natl. Acad. Sci. USA., 1972; 69: 3561-3566).

In another embodiment of the invention, the membrane may comprise a solid state layer. Solid state layers can be formed from organic and/or inorganic materials including, but not limited to, microelectronic materials, insulating materials such as Si₃N₄, Al₂O₃, and SiO, glasses, organic and inorganic polymers such as polyamide and plastics. The solid state layer may be formed from graphene such as disclosed in PCT/US2008/010637. The membrane may be provided with one or more apertures or through-holes of nanometre scale dimensions extending from one side of the membrane to the other. The internal walls of the solid state aperture may be coated with a lipid such as disclosed in US2017/0023544 or chemically functionalized such as disclosed in PCT/US2008/063066, so as to facilitate suitable anchoring of the nanostructures of the invention to the solid state layer.

A nanopore according to an embodiment of the present invention is a membrane or solid state substrate spanning nanostructure that is embedded within and, at least a portion thereof, is oriented substantially coplanar to a membrane or partition surface. In an embodiment of the invention, the nanopore is located in a membrane or substrate in a manner akin to a grommet or eyelet mounted in a planar or curved sheet material. Hence, in a specific embodiment of the invention the nucleic acid nanostructure of the invention is defined as a nanoscale grommet or eyelet, this is irrespective of the shape of the channel(s) which may be circular or polygonal. According to this embodiment of the invention a majority of the nanostructure of the nanopore is embedded within the membrane compared to the proportion of the nanostructure extending outside of the membrane. In a further embodiment of the invention the nanostructure comprises one or more modules that may extend radially from one or both sides of the pore but which are substantially co-planar with and sit upon a surface of the membrane.

Suitably the nanopores of the invention comprise one or more polynucleotide strands that provide a functional scaffold component, wherein the polynucleotide strands comprised within the scaffold component include a polynucleotide backbone; and a plurality of polynucleotide strands that provide a plurality of functional staple components. The scaffold strand(s) cooperate with and hybridise to the plurality of staple polynucleotide strands—e.g. via appropriate Watson-Crick base pairing hybridisation—in order to form a three-dimensional configuration of the nanopore. It is desirable that the nanopore assembles into an annular, elliptical, regular or irregular polygonal structure that is able to be embedded within a membrane or partition surface such that a majority of the 5′ to 3′ orientation of the staple component(s) is coplanar with the membrane. Hence within modules embedded within or coplanar with—e.g. lying upon—the membrane a majority of the 5′ to 3′ orientation of the staple component(s) of the modules are coplanar with the orientation of the membrane.

Alternatively, a nanopore according to a further embodiment of the invention may comprise a DNA nanostructure such as a nanobarrel or nanoraft, which is typically a rectangular, regular or irregular polygonal, circular, or ellipsoid substantially planar nanostructure. In this configuration the portion of the nanopore that spans the membrane has a 5′ to 3′ or 3′ to 5′ orientation that is substantially perpendicular to the plane of the membrane. Hence, the nucleic acid duplexes are formed into a bundle, or a series of modules comprised of bundles of duplexes, that extend through the membrane thereby forming the nanostructure that defines the pore. Analyte sensing may be enhanced by the installation of a molecular receptor within the pore lumen or channel.

Nanomechanically actuatable nucleic acid nanopores having this perpendicular configuration may have a central channel or lumen with a relatively large minimum diameter, for example, a minimum internal width greater than about 5 nm. The lumen is surrounded and defined by a generally elongate cylindrical pore wall. The nanopore comprises two regions: a cap region and a membrane-spanning region. The membrane-spanning region is defined as the portion of the nanopore located within the plane of the membrane, and the cap region being the portion of the nanopore attached to the membrane-spanning region and extending away from the surface of the membrane, typically on the cis side of the membrane. The nanopore may have a cap region on one side of the membrane only, or alternatively, have two cap regions, one on each side of the membrane. When there is more than one cap region, these may be the same as each other or they may be different. Suitably, the nanopore has one cap region on one side of the membrane forming an entrance to the nanopore.

The cap region may have dimensions of any suitable size. While it is possible for the cap region to extend only negligibly from the membrane surface, typically, the cap region has a height extending from the membrane of at least 5 nm as measured by the perpendicular distance from the membrane surface to the top of the pore wall. Suitably, the cap region may have a height of at least 10 nm, 15 nm, 20 nm, 25 nm, 30 nm, 35 nm, 40 nm, 45 nm or 50 nm or above. Suitably, the height of the cap region is at most 100 nm, 90 nm, 80 nm, 70 nm, 60 nm, or 50 nm or below. The height of the cap region may be determined by the length of the scaffold polynucleotide used, and the number of layers of polynucleotide duplexes that form the pore. For example, according to calculations using computer software (CaDNAno software, available at http://www.cadnano.org; or the DAEDALUS online platform, available at http://daedalus-dna-origami.org), for a square cross-section DNA pore using M13mp18 or a phix174 scaffold strand and with a minimum interior width of the pore of 20 nm, the maximum height of the pore is around 37 nm when the pore wall is two duplexes thick; 20 nm when the pore wall is three duplexes thick; and 13 nm when the pore is four duplexes thick.

The membrane-spanning region may have dimensions of any suitable size. Typically, the membrane-spanning region has a height that approximately matches the thickness of the membrane in which it resides. The thickness of biological lipid bilayer membranes can range from around 3.5 to 10 nm. The thickness of membrane composed of amphiphilic synthetic block copolymers shows a wider range from 5 to 50 nm (C. LoPresti, H. Lomas, M. Massignani, T. Smart, G. Battaglia, J. Mater. Chem. 2009, 19, 3576-3590). Therefore suitably, the membrane-spanning region may have a height of at least around 3.5 nm, although it may be possible to have a membrane-spanning region with a height as low as 3 nm, 2.5 nm, 2 nm 1.5 nm or 1.0 nm or less. Suitably, the membrane-spanning region may have a height of at least 5 nm. The membrane-spanning region may have a height of at most 100 nm, 90 nm, 80 nm, 70 nm, 60 nm, 50 nm, 40 nm, 30 nm, 20 nm or 10 nm or less. Suitably the membrane-spanning region has a maximum height of 50 nm for synthetic polymer layers, and a maximum height of 10 nm for lipid bilayers.

The actuatable nanopores of all configurations of the present invention may be assembled via the ‘scaffold-and-staple’ approach. In this important route to nucleic acid nanostructures, DNA is utilized as a building material in order to make nanoscale three dimensional shapes. Assembly of these complex nanostructures from a plurality of un-hybridized linear molecules is typically referred to as ‘DNA origami’. The DNA origami process generally involves the folding of the one or more elongate, ‘scaffold’ DNA strands into a particular shape using a plurality of rationally designed ‘staple’ DNA strands. The scaffold strand can have any sufficiently non-repetitive sequence. The sequences of the staple strands are designed such that they hybridize to particular defined portions of the scaffold strands and, in doing so, these two components cooperate force the scaffold strands to assume a particular structural configuration. Methods useful in the making of DNA origami structures can be found, for example, in Rothemund, P. W., Nature 440:297-302 (2006); Douglas et al, Nature 459:414-418 (2009); Dietz et al, Science 325:725-730 (2009); and U.S. Pat. App. Pub. Nos. 2007/0117109, 2008/0287668, 2010/0069621 and 2010/0216978, each of which is incorporated by reference in its entirety. Staple sequence design can be facilitated using, for example, CaDNAno software, available at http://www.cadnano.org or the DAEDALUS online platform, available at http://daedalus-dna-origami.org.

In embodiments of the invention the staple and/or scaffold components further comprise a plurality of hydrophobic membrane anchor molecules that are attached thereto. The hydrophobic anchors (or portions of the sequence) facilitate insertion of the nanopore into a curved or planar membrane such that the orientation of a major portion of the first scaffold polynucleotide strand is substantially parallel to the surface of the membrane and wherein the first scaffold polynucleotide strand is embedded within and is substantially coplanar with the membrane. In referring to ‘a major portion’ of a scaffold polynucleotide stand it is meant that substantially more than 50%, suitably more than 60%, even greater than 70%, and as much as 90%, of the total length of that strand is orientated so that it is substantially coplanar with the membrane. In a specific embodiment the entire length of the scaffold strand, excepting crossovers and/or Holliday junctions necessary to impart three-dimensional structure on a resultant nanopore, is orientated so that it is substantially coplanar with the membrane. It will be appreciated that DNA origami techniques allow for variations of the embodied structures that, nevertheless, fall within the overall design constraints of the recited nanostructures of the present invention. By way of example, the scaffold strand may be comprised of a plurality of shorter scaffold strands that, when assembled following hybridisation to appropriate staple strands, will cooperate to serve in a manner equivalent to a unitary single length scaffold strand.

In embodiments, the nanopores of the present invention are formed or constructed from one or more modules. In embodiments, the nanopore may be formed of an arrangement of modules that forms a basic frame or framework. In embodiments, the modules of the frame are supported by additional, typically smaller, sub-modules that connect and support the structure of the frame. At least part of the module is intended to form at least part of the channel wall of the nanopore and therefore the module is designed and configured to span a membrane in which it is inserted. Sub-modules are intended for structural benefits only and therefore are not intended or configured to span the membrane. While each module of the frame, or the sub-modules, may be different, suitably, the modules of the frame are suitably substantially or completely identical units, as are the sub-modules forming the support, when present. In these embodiments, the modules and sub-modules are each formed of the same scaffold and staple DNA structure and assembled in the same way.

The individual modules may be joined by DNA strands, the DNA strand either being integral with the module, or hybridised to each module. While any arrangement of the modules is contemplated, suitably, the modules may be arranged to overlie each other to form a generally hollow stack or tower thereby forming a channel. Suitably, for nanopores having a polygonal cross-section, the modules are arranged such that they sit side by side in the plane of a membrane. The modules may have tuneable side length (a side length in this context being defined as the longest dimension of the module parallel to the plane of the membrane), which when chosen with an appropriate final overall shape, allows for different sized and/or shaped nanopores, and different sized and/or shaped lumens within the channels of those pores to be prepared. The channels and the lumens defined thereby may be regularly or irregularly shaped. For example, the lumens defined by the channel may be a regular or irregular polygon, such as a triangle, a quadrilateral (e.g. a square, a rectangle or a trapezoid), a pentagon, a hexagon, a heptagon, an octagon and so on. Alternatively, the channels may be an elongate polygon, such as rectangular, oblong or slot-shaped channels formed of 4 or more sides. Typically, the side length of the modules would be in the order of between 10 nm and 20 nm. Suitably, the side length of the modules may be at least 2 nm, 3 nm, 4 nm, 5 nm, 6 nm, 7 nm, 8 nm, 9 nm or 10 nm, Suitably the side length of the modules may be at most 30 nm, 25 nm, 20 nm. The sizing of the sub-modules is determined by the spacing between the modules which is turn is determined by the shape of the pore and the size and number of modules employed. Suitably, the side length of the modules may be at least 0.5 nm, 1 nm, 1.5 nm, 2 nm, 2.5 nm, 3 nm, 3.5 nm, 4 nm or 5 nm, Suitably the side length of the modules may be at most 10 nm, 7.5 nm, or 5 nm.

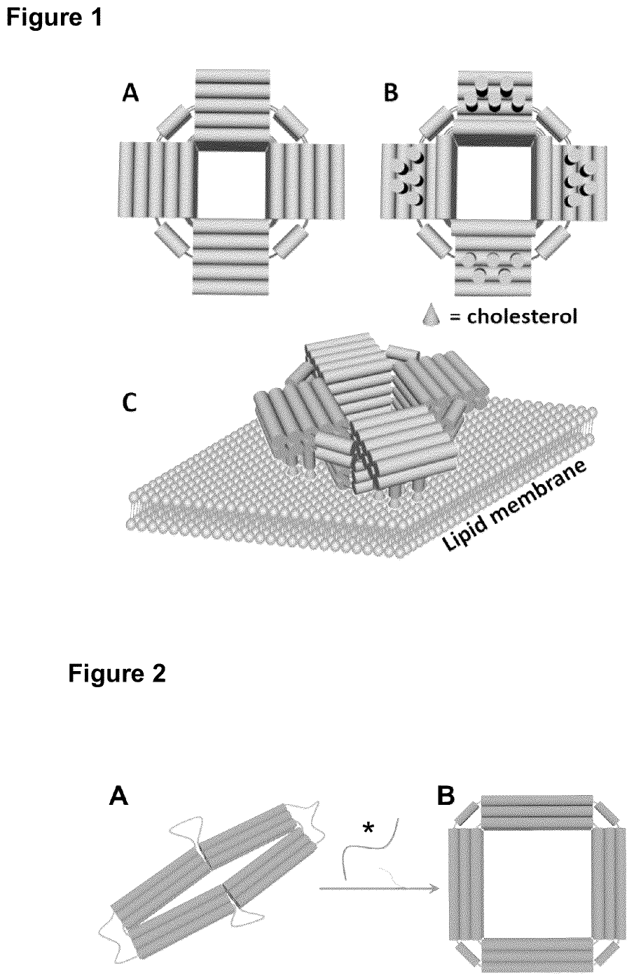

The internal corners between adjacent walls or modules within the lumen of the nanopore are referred to as vertices. Depending upon the nature of the polygonal format selected nanomechanical actuation may occur around flexure of one or more vertices. Hence, the movement occurring around these regions of the nanopore is hinge-like. Typically, at least a pair of vertices will be capable of flexure in response to an external stimulus thereby resulting in conformational change of the nanopore structure that, in turn, results in a change in the cross sectional area of the lumen. In specific embodiments, the vertices capable of flexure are located on opposing sides of a nanopore as shown in FIG. 2.

As used herein, the term ‘substantially’ refers to the complete or nearly complete extent or degree of an action, characteristic, property, state, structure, item, or result. For example, an object that is ‘substantially’ coplanar with another object would mean that the object is either completely coplanar or nearly completely coplanar, perhaps varying by a few degrees of variation from complete conformity. The exact allowable degree of deviation from absolute completeness may in some cases depend on the specific context, as would be understood to the person of skill in the art. However, in general terms the nearness of conformity to the absolute will be such as to have the same overall result—e.g. functional equivalence—as if total conformity were achieved. Hence, where a nanopore of the invention assumes a substantially expanded conformation, it is understood to mean that the nanopore is completely or nearly completely radially expanded.

As shown in FIG. 1, the three-dimensional configuration of the nanopores (10) of the present invention defines at least one channel (11), suitably a single channel that spans the membrane, the channel having a lumen that has a minimum internal width of at least about 3 nm. Suitably, the nanopores of the present invention have a single channel located at least substantially centrally in the pore structure when viewed perpendicular to the plane of the membrane in which the pore is intended to reside. The channel defines the lumen that passes through the nanopore which is perpendicular to the planar axis defined by the membrane. The minimum opening, or aperture, of the channel in this cross-section (e.g. the minimum constriction) is suitable to facilitate a close fitting interaction with a folded protein or other analyte. Typically, the minimum opening is at least 3 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, or 15 nm or more. Suitably the lumen is between around 10 nm and around 20 nm in width. Suitably, the maximum opening of the lumen channel (i.e. minimum constriction) is at most 200 nm, 150 nm, 100 nm, 75 nm, 50 nm, 40 nm, 30 nm, 20 nm, 18 nm, 15 nm, 12 nm, or 10 nm. Suitably, the cross-sectional area of the lumen of the channel (i.e. minimum constriction) in at least one conformation is at least 5 nm², 12 nm², 15 nm², 25 nm², 35 nm², 40 nm², 45 nm² or 50 nm² or more. Suitably, the cross-sectional area of the lumen of the channel (i.e. minimum constriction) in at least one conformation is at most 35 000 nm², 25 000 nm², 15 000 nm², 10 000 nm², 5 000 nm², 1500 nm², 1000 nm², 750 nm², 500 nm², 250 nm², 100 nm², 50 nm², 30 nm², 20 nm² or 15 nm², 10 nm², 7 nm² or less. The transition from at least a first conformation to at least a second conformation results in a corresponding change in the cross sectional area of the lumen. This, in turn results in a change in any electrical current passing through the lumen which can be measured.

In an embodiment of the present invention the nanopores are provided with at least one vertex between adjacent wall-defining modules that exhibit a high level of flexure. This manifests as a hinging action and results in lateral compression of the nanopore in response to the radial forces exerted by the surrounding membrane. Typically, the nanopore will comprise at least two vertices capable of hinging, optionally these may be located on opposing sides of the nanopore to allow the nanopore to constrict the central lumen and reduce the luminal cross-sectional area. It will be understood that the geometry of the nanopores of the invention is such that complete closure is unlikely to occur with the lumen tending to form an elongate slit or letterbox configuration.

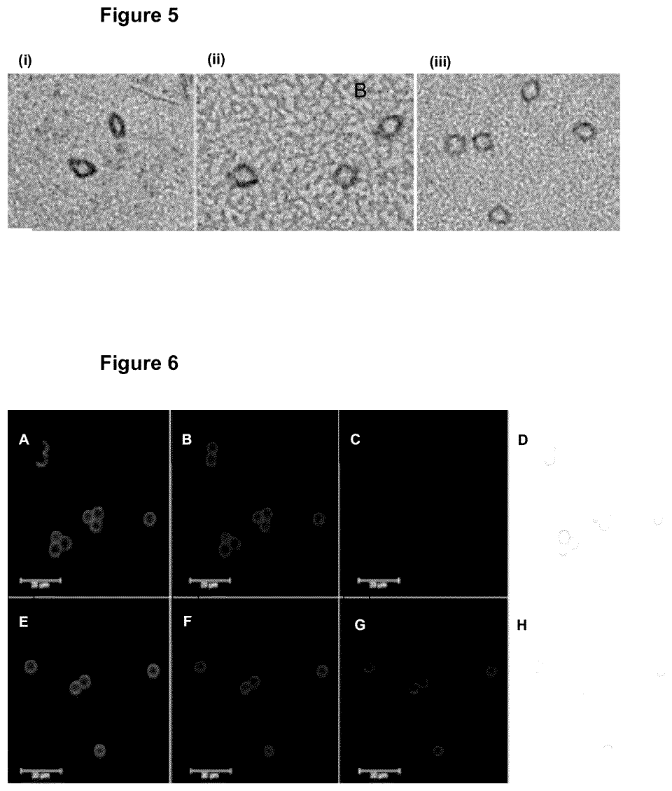

The nanopores of the present invention are provided with a trigger region that acts as a mechanism for facilitating the transition of the nanopore from at least a first conformation of lower cross sectional area to at least a second conformation of higher cross sectional area. It will be appreciated that depending upon the precise polygonal configuration of the pore, there may be one or more intermediate transitional conformations that exist between the first and second conformations. In a specific embodiment of the invention, the trigger mechanism is comprised of a region of at least one single stranded nucleic acid within a linking structure between the adjacent modules that form the walls of the nanopore. This region of single stranded nucleic acid lacks the rigidity of a double helix and contributes to the properties of flexure required to ensure that the vertex is capable of behaving like a hinge such that in the absence of a stimulus the nanopore is in the first configuration. This region, termed the trigger region, is readily accessible to the surrounding solution such that target analyte present in solution may bind to the trigger region, thereby providing the stimulus, and lead to a change in its structural conformation. Such a change in structural conformation leads to initiation of a trigger event causing the nanopore as a whole to switch from first to second conformation. For example, in one embodiment of the invention the analyte is a target single stranded nucleic acid present in solution (e.g. a single stranded nucleic acid sequence such as a viral genomic RNA, or fragment thereof) which is capable of hybridising to the trigger region. The binding of the one or more target nucleic acid molecules to the trigger region results in hybridisation and formation of the conventional double helical structure through Watson-Crick base pairing. This increases steric repulsive forces within the nanostructure modules which exceed the radial compression from the surrounding membrane, thereby causing the nanopore to transition to a more open second configuration. In an alternative embodiment, the trigger region may comprise a linker to a binding moiety. The binding moiety may be linked covalently to the single stranded nucleic acid of the trigger region, such as via a nitriloacetate (NTA) conjugation, which has high affinity to a His-tagged binding moiety via complexation of Ni²⁺ or Co³⁺ (Shimada et al. (2008) Biotechnology Letters, (30):2001-2006). Upon binding of the analyte to the binding moiety steric effects in the trigger region can result in initiation of a trigger event causing the nanopore as a whole to switch from a first to a second conformation—with a corresponding change in lumenal cross-sectional area. Thus, in accordance with the present invention, this nanostructural conformational change provides an increased rigidity and resistance to the radial compressive forces resulting from lateral pressure of the membrane. The radial expansion of the nanopore results in a nanostructural switch from a first conformation (compressed) to a second conformation (expanded), and can be seen in TEM images (FIG. 5).

A target nucleic acid sequence may represent an external stimulus and may serve as a way of activating the trigger mechanism within the nucleic acid nanostructure. Suitably, the target nucleic acids may be oligonucleotides or polynucleotides, that are naturally occurring (e.g. ssDNA or ssRNA) or of synthetic origin.

The binding moiety may comprise a polynucleotide or a polypeptide that is capable of binding to an analyte present in a solution that surrounds the membrane-embedded nanopore. Where the binding molecule comprises a polypeptide that is tethered to the nanopore, either within the lumen or proximate to the cis or trans side of the nanopore, it may be selected from one of the group consisting of:
I. an enzyme—including a polymerase, a helicase, a gyrase, and a telomerase, as well as nucleic acid binding sub domains or derivatives thereof
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including, molecular tags (e.g. biotin, polyhistidine-tag), drug molecules (e.g. small molecules), fluorophores, metabolites and chemokines;
VI. an antigen or antigenic fragment; and
VII. signalling molecules and/or polypeptide receptors thereof, including binding domains of receptors, and receptor complexes.

Alternatively, the affinity binding molecule may comprise a small molecule, a lipid group, a polysaccharide group, a polymer or any other molecule naturally-occurring or synthetic molecule that is capable of effecting a specific affinity binding interaction with an analyte in solution.

The one or more binding moieties may be attached to the pore via a covalent or non-covalent linkage, such as via avidin-biotin or His-tag type interaction—e.g. via NTA linkage.

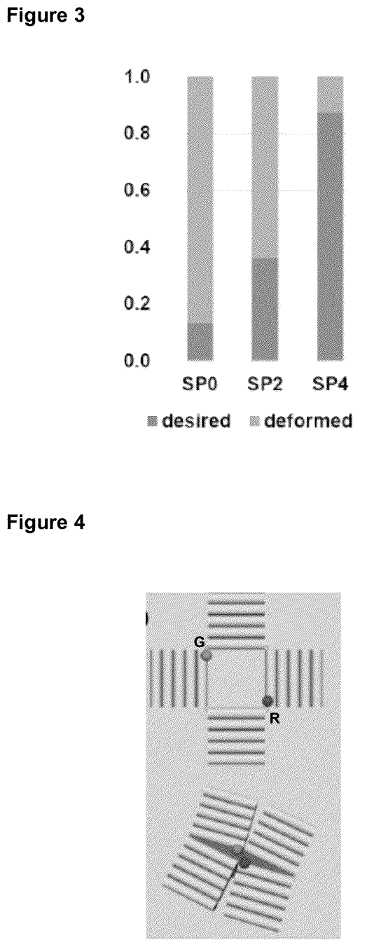

One advantage of the nanomechanical pores described herein is that they allow for more than one recognition site to be included within the pore thereby improving the specificity of the interaction. For example, polygonal pores having four or more sides may include multiple recognition sites, i.e. one per trigger region. By way of further example, a pore having a square configuration (such as the pore shown in FIGS. 1 and 2) will comprise two trigger regions at opposing apices. A first trigger region may be responsive to a first stimulus and the second trigger region may be responsive to a second stimulus. The first and second stimuli may be the same or different. Hence in response to the first stimulus (e.g. an analyte binding event) the first trigger region will change in conformation but the nanomechanical structure will not fully transition from first to second conformations, but will adopt an intermediate partially expanded state. This is range of transition is shown in FIG. 3, where SP0 state is the first conformation having a lower cross sectional lumenal surface area (compressed), SP2 is the transitional configuration upon binding of a first stimulus and SP4 represents second conformation having a greater cross sectional lumenal surface area (expanded) where first and second stimulus are present. It will be appreciated that the proportion of a number of pores in the SP0 or SP4 states will not correspond to 0 or 100% (shown as a fraction of 1.0 on the y axis of FIG. 3) respectively due to dynamic equilibrium (Le Chatelier's principle) such that at any one time there will always be a small proportion of the nanostructures that are transitioning from one conformation state to another.

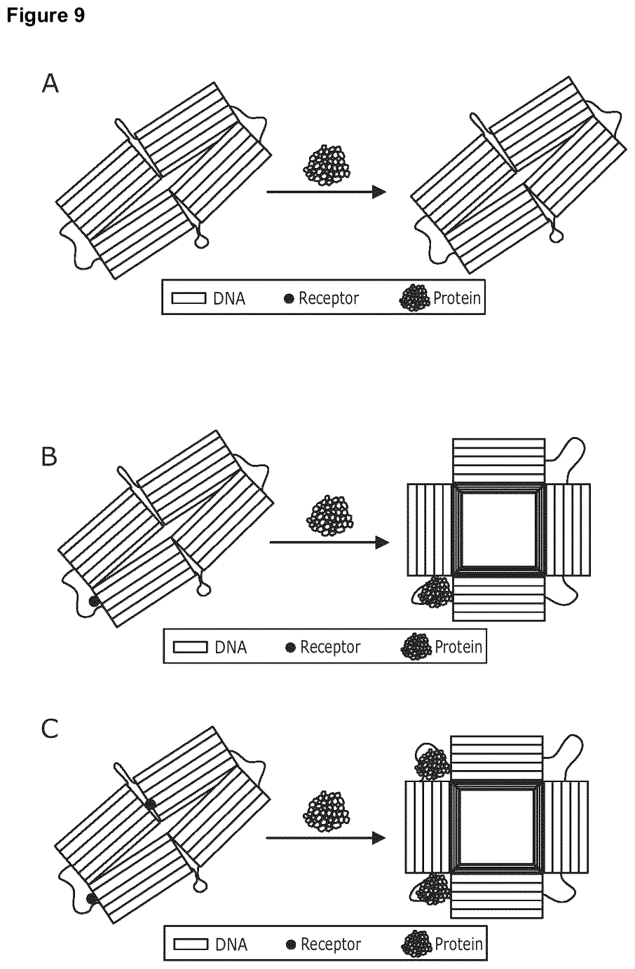

In an alternative embodiment of the invention shown in FIG. 9A-C, polygonal pores having four or more sides may include multiple recognition sites located at adjacent apices. FIG. 9C shows an exemplary pore having a square configuration which comprises two trigger regions that are functionalised with a polypeptide binding moiety situated at adjacent apices. The first and second stimuli may be the same polypeptide analyte or different. Hence in response to the first stimulus (e.g. an analyte binding event) the first trigger region will change in conformation from first to second confirmation. It will be appreciated that the nanomechanical structure may not fully transition from first to second conformations but may adopt an intermediate partially expanded state as described previously.

In classical nanopore biosensing, a single molecular binding moiety, such as a tethered receptor or antibody, is sufficient to achieve binding of the analyte into the pore lumen and causing a detectable current blockade. In this approach a single binding event results in binary output of a single detectable signal, either the analyte is bound or not bound (e.g. 1 or 0). By comparison, a nanomechanical pore of the type described herein allows a designer to engineer a plurality of analyte recognition sites that are located at different parts of the pore—as described previously—not just within the lumen. One advantage of this approach is that an analyte binding event at each recognition site can represent a discrete stimulus that initiates the trigger for nanomechanical transition of the pore from one conformation to another. The ability to engineer nanomechanical pores with a plurality of analyte recognition sites expands the range of biosensing to allow for recognition of more than one analyte, for example where each recognition site recognises a different analyte present in the sample. Alternatively, where all recognition sites recognise the same analyte, the plurality of sites allows for some determination of the concentration of the analyte in the solution as a factor of recognition site occupancy. The nanostructures presently described may be engineered, in specific embodiments, to recognise a range of different analytes, including nucleic acids and proteins, within single sample. For example, the present invention makes it possible to provide simultaneous nanopore sensing for both nucleic acid and polypeptide components from a biological sample comprising infectious disease pathology—e.g. virus capsid proteins and/or virus genomic nucleic acid, such as SARS-COV-2 spike protein and/or RNA genome.

A further advantage of the nanomechanical pores of the present invention is that there is a decoupling of the relationship between signal and analyte size that is typically required for conventional nanopore sensors. Where an analyte is bound within or proximate to the lumen, this results in a blockade of an electrical signal, such as current, passing through the lumen. If the analyte is very small then the amount of obstruction, and therefore the degree of change of signal, may be low. The nanomechanical pores of the present invention can act as signal amplifiers in that the binding of an analyte triggers the change in conformation that results in the corresponding change in signal output, for example a significant change in measurable electrical current through or across the pore, or a loss of a FRET signal. Hence the amplitude of the signal output is independent of the size of the analyte, thereby expanding the range of sensor applications that these nanopores can be applied to.

The nanopore structures of the present invention may be used in sensor applications that allow for the detection of a diverse range of potential analytes that may exist in a solution that is under test. Exemplary analytes may include:
peptides/polypeptides/proteins—folded, partially/completely unfolded;
enzymes;
protein/nucleic acid constructs;
molecules defined by size;
macromolecules within specified size ranges, for example, in ranges selected from: 1-10 kD, 1-50 kD, 1-100 kD, 10-50 kD, 10-100 kD, 20-50 kD, and 20-100 kD;
multi-protein complexes;
antigens or their antibodies;
virion particles and bacterial cells
glycoproteins;
carbohydrates;
biopolymers;
toxins;
metabolites and by products thereof;
cytokines;
nucleic acids

The nanopore structures of the present invention may be incorporated within a plurality of improved devices and sensors. Such devices and sensors are useful in applications requiring to sensing and characterization of a variety of materials and analytes. By way of non-limiting example, particularly useful applications, including genome sequencing, protein sequencing, other biomolecular sequencing, and detection of ions, molecules, chemicals, small molecules, biomolecules, metal atoms, contaminants, polymers, nanoparticles etc. Such detecting and characterizing can, in turn, be used to diagnose diseases, in drug development, to identify contamination or adulteration or food or water supplies, and in quality control and standardization.

According to exemplary embodiments of the present invention as described, a sensor device typically comprises a substrate that includes a membrane partition into which one or more nanopores are embedded. The substrate is placed to facilitate contact with a fluid (optionally an electrolytic solution) which comprises an analyte. At least one, and optionally a plurality of, device(s) are positioned relative to the substrate, wherein a given device generates a signal (e.g. mechanical, electrical, and/or optical) in response to detecting binding to and/or passage through the nanopore(s) of one or one or more analytes. The plurality of devices can be greater than 2 and as many as 100, or as many as 20, or as many as 10, or between 2 and 8 devices. Each device may be selected from one or more of the group consisting of: a field effect sensor; a plasmonic sensor; a laser based sensor; an interferometric sensor; a wave-guide sensor; a cantilever sensor; an acoustic sensor; a quartz crystal microbalance (QCM) sensor; an ultrasonic sensor; a mechanical sensor; a thermal sensor; an optical dye based sensor; a fluorimetric sensor; a calorimetric sensor; a luminometric sensor; a graphene sensor; a quantum dot sensor; a quantum-well sensor; a photoelectric sensor; a 2D material sensor; a nanotube or nanowire sensor; an enzymatic sensor; an electrochemical sensor, including a FET or BioFET sensor; a potentiometric sensor; a conductometric sensor; a capacitive sensors; and an electron-spin sensor. The devices may cooperate in the form of arrays allowing for multiplexed testing of multiple analytes. The sensor devices may further comprise special purpose hardware and systems (e.g., circuitry, processors, memory, GUIs etc.) that perform the specified functions or acts, or combinations of special purpose hardware and computer instructions, in order to render a functioning sensor device capable of providing a meaningful readout to a user.

Hence, according to embodiments of the invention suitably configured nanopore devices may enable a variety of different types of sensor measurements to be made. Typically, electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with or based upon electrical measurements (Soni G V et al., Rev Sci Instrum. 2010 January; 81(1):014301), for instance, via conversion of ionic current into a fluorescent signal from an indicator dye (e.g. Fluo-8) arising from Ca²⁺ flux through a nanopore (Huang et al. Nat Nanotechnol. 2015 November; 10(11): 986-991). As mentioned previously, the measurement may be a transmembrane current or voltage measurement such as measurement of ionic current flowing through the nanopore. Alternatively, the signal may be obtained from measurement of a change in transverse membrane current, voltage and/or impedance value over time. Hence, a conformational change in the nanopore will result in a detectable output signal that can be measured.

In a specific embodiment of the invention, as shown in FIG. 4, opposing vertices of the nanopore may be labelled with one or more electromagnetic radiation responsive tags such as fluorophores, chromophores or quantum dots. In the contracted state, the tags may be relatively close together allowing for resonance energy transfer (e.g. fluorescence resonance energy transfer (FRET)) that is detectable with an external photodetector or light sensor (e.g. CCD or photodiode). Upon actuation the conformational change as the nanopore expands radially increases the distance between tags thereby interrupting resonance energy transfer between tags and changing or eliminating the detectable output signal from the nanopore. This effect is shown in FIG. 6, where mechanically actuatable nanopores labelled with a FRET dye pair, green-Atto565 (G) and red-Atto647 (R), are located at opposing vertices of the nanopore. Upon binding of an analyte to the nanopore FRET is disrupted (FIG. 6G shows fluorescence prior to analyte binding, which is lost following conformation shift to the second expanded conformation in FIG. 6C).

Hence in accordance with embodiments of the present invention methods for sensing the presence of an analyte, suitably an analyte comprised within a liquid sample, comprise:
I. providing a sensor device as described herein;
II. contacting a nanopore comprised within the sensor device with an analyte and establishing a flow of ions through the nanopore or an electron flow across the nanopore; and
III. measuring an electrical signal across the nanopore,
wherein the sensing comprises analyte detection or characterisation, wherein a change in the electrical measurement is indicative of the presence of the analyte.

In alternative embodiments methods for sensing the presence of an analyte may comprise:
(a) providing a sensor device as described herein, wherein the device is configured for FRET detection with photodetector or light sensor;
(b) contacting a nanopore comprised within the sensor device with an analyte that acts as a stimulus to initiate a conformational change of the nanopore from a first conformation to at least a second conformation; and
(c) measuring an output signal response via a fluorescence resonance energy transfer (FRET) technique.

The invention is further illustrated by the following non-limiting examples.

### EXAMPLES

### Example 1—Detection of Nucleic Acid Analytes

A square nucleic acid membrane-bound nanopore which defines a correspondingly square lumen when in second conformation was used as a test nanopore.

The pore was selected to have a 20 nm side length, thereby providing a maximal cross section lumenal surface area of 400 nm² when the pore is the second conformation, and a smaller surface area when in the first conformation. The pore was constructed via DNA origami using a phix174 scaffold and corresponding staple nucleic acid sequences according to methods substantially as described in WO-2020/025974-A. The pore was of the type shown in FIG. 8, except that two opposing vertices have inner and outer single stranded (SS) DNA that define the trigger regions. The remaining two vertices have the SS and DS DNA configuration as shown in FIG. 8. This results in the nanopore structure adopting the configuration having reduced lumenal cross sectional area upon insertion into a membrane as shown in FIG. 2A. The phix174 scaffold sequence is provided in Table 1 below with parts of the scaffold that contribute to the trigger regions highlighted in bold and underlined:

| | |
|---|---|
| phiX174 | CCTGCAGAGTTTTATCGCTTCCATG |
| (SEQ ID | ACGCAGAAGTTAACACTTTCGGATATTTCTGATGAG |
| NO: 1) | TCGAAAAATTATCTTGATAAAGCAG |
| | GAATTACTACTGCTTGTTTACGAATTAAATCGAAGT |
| | GGACTGCTGGCGGAAAATGAGAAAATTCGACCTATC |
| | CTTGCGCAGCTCGAGAAGCTCTTACTTTGCGACCTT |
| | TCGCCATCAACTAACGATTCTGTCAAAAACTGACGC |
| | GTTGGATGAGGAGAAGTGGCTTAATATGCTTGGCAC |
| | GTTCGTCAAGGACTGGTTTAGATATGAGTCACATTT |
| | TGTTCATGGTAGAGATTCTCTTGTTGACATTTTAAA |
| | AGAGCGTGGATTACTATCTGAGTCCGATGCTGTTCA |
| | ACCACTAATAGGTAAGAAATCATGAGTCAAGTTACT |
| | GAACAATCCGTACGTTTCCAGACCGCTTTGGCCTCT |
| | ATTAAGCTCATTCAGGCTTCTGCCGTTTTGGATTTA |
| | ACCGAAGATGATTTCGATTTTCTGACGAGTAACAAA |
| | GTTTGGATTGCTACTGACCGCTCTCGTGCTCGTCGC |
| | TGCGTTGAGGCTTGCGTTTATGGTACGCTGGACTTT |
| | GTAGGATACCCTCGCTTTCCTGCTCCTGTTGAGTTT |
| | ATTGCTGCCGTCATTGCTTATTATGTTCATCCCGTC |
| | AACATTCAAACGGCCTGTCTCATCATGGAAGGCGCT |
| | GAATTTACGGAAAACATTATTAATGGCGTCGAGCGT |
| | CCGGTTAAAGCCGCTGAATTGTTCGCGTTTACCTTG |
| | CGTGTACGCGCAGGAAACACTGACGTTCTTACTGAC |
| | GCAGAAGAAAACGTGCGTCAAAAATTACGTGCAGAA |
| | GGAGTGATGTAATGTCTAAAGGTAAAAAACGTTCTG |
| | GCGCTCGCCCTGGTCGTCCGCAGCCGTTGCGAGGTA |
| | CTAAAGGCAAGCGTAAAGGCGCTCGTCTTTGGTATG |
| | TAGGTGGTCAACAATTTTAATTGCAGGGGCTTCGGC |
| | CC |
| | CTTACTTGAGGATAAATTATGTCTA |
| | ATATTCAAACTGGCGCCGAGCGTATGCCGCATGACC |
| | TTTCCCATCTTGGCTTCCTTGCTGG |
| | TCAGATTGGTCGTCTTATTACCATTTCAACTACTCC |
| | GGTTATCGCTGGCGACTCCTTCGAGATGGACGCCGT |
| | TGGCGCTCTCCGTCTTTCTCCATTGCGTCGTGGCCT |
| | TGCTATTGACTCTACTGTAGACATTTTTACTTTTTA |
| | TGTCCCTCATCGTCACGTTTATGGTGAACAGTGGAT |
| | TAAGTTCATGAAGGATGGTGTTAATGCCACTCCTCT |
| | CCCGACTGTTAACACTACTGGTTATATTGACCATGC |
| | CGCTTTTCTTGGCACGATTAACCCTGATACCAATAA |
| | AATCCCTAAGCATTTGTTTCAGGGTTATTTGAATAT |
| | CTATAACAACTATTTTAAAGCGCCGTGGATGCCTGA |
| | CCGTACCGAGGCTAACCCTAATGAGCTTAATCAAGA |
| | TGATGCTCGTTATGGTTTCCGTTGCTGCCATCTCAA |
| | AAACATTTGGACTGCTCCGCTTCCTCCTGAGACTGA |
| | GCTTTCTCGCCAAATGACGACTTCTACCACATCTAT |
| | TGACATTATGGGTCTGCAAGCTGCTTATGCTAATTT |
| | GCATACTGACCAAGAACGTGATTACTTCATGCAGCG |
| | TTACCATGATGTTATTTCTTCATTTGGAGGTAAAAC |
| | CTCTTATGACGCTGACAACCGTCCTTTACTTGTCAT |
| | GCGCTCTAATCTCTGGGCATCTGGCTATGATGTTGA |
| | TGGAACTGACCAAACGTCGTTAGGCCAGTTTTCTGG |
| | TCGTGTTCAACAGACCTATAAACATTCTGTGCCGCG |
| | TTTCTTTGTTCCTGAGCATGGCACTATGTTTACTCT |
| | TGCGCTTGTTCGTTTTCCGCCTACTGCGACTAAAGA |
| | GATTCAGTACCTTAACGCTAAAGGTGCTTTGACTTA |
| | TACCGATATTGCTGGCGACCCTGTTTTGTATGGCAA |
| | CTTGCCGCCGCGTGAAATTTCTATGAAGGATGTTTT |
| | CCGTTCTGGTGATTCGTCTAAGAAGTTTAAGATTGC |
| | TGAGGGTCAGTGGTATCGTTATGCGCCTTCGTATGT |
| | TTCTCCTGCTTATCACCTTCTTGAAGGCTTCCCATT |
| | CATTCAGGAACCGCCTTCTGGTGATTTGCAAGAACG |
| | CGTACTTATTCGCCACCATGATTATGACCAGTGTTT |
| | CCAGTCCGTTCAGTTGTTGCAGTGGAATAGTCAGGT |
| | TAAATTTAATGTGA |
| | CCGTTTTATCGCAATCTGCCGACCAC |
| | TCGCGATTCAATCATGACTTCGTGATAAAAGATTGA |
| | GTGTGAGGTTATAACGCCGAAGCGG |
| | TAAAAATTTTAATTTTTGCCGCTGAGGGGTTGACCA |
| | AGCGAAGCGCGGTAGGTTTTCTGCTTAGGAGTTTAA |
| | TCATGTTTCAGACTTTTATTTCTCGCCATAATTCAA |
| | ACTTTTTTTCTGATAAGCTGGTTCTCACTTCTGTTA |
| | CTCCAGCTTCTTCGGCACCTGTTTTACAGACACCTA |
| | AAGCTACATCGTCAACGTTATATTTTGATAGTTTGA |
| | CGGTTAATGCTGGTAATGGTGGTTTTCTTCATTGCA |
| | TTCAGATGGATACATCTGTCAACGCCGCTAATCAGG |
| | TTGTTTCTGTTGGTGCTGATATTGCTTTTGATGCCG |
| | ACCCTAAATTTTTTGCCTGTTTGGTTCGCTTTGAGT |
| | CTTCTTCGGTTCCGACTACCCTCCCGACTGCCTATG |
| | ATGTTTATCCTTTGGATGGTCGCCATGATGGTGGTT |
| | ATTATACCGTCAAGGACTGTGTGACTATTGACGTCC |
| | TTCCCCGTACGCCGGGCAATAATGTTTATGTTGGTT |
| | TCATGGTTTGGTCTAACTTTACCGCTACTAAATGCC |
| | GCGGATTGGTTTCGCTGAATCAGGTTATTAAAGAGA |
| | TTATTTGTCTCCAGCCACTTAAGTGAGGTGATTTAT |
| | GTTTGGTGCTATTGCTGGCGGTATTGCTTCTGCTCT |
| | TGCTGGTGGCGCCATGTCTAAATTGTTTGGAGGCGG |
| | TCAAAAAGCCGCCTCCGGTGGCATTCAAGGTGATGT |
| | GCTTGCTACCGATAACAATACTGTAGGCATGGGTGA |
| | TGCTGGTATTAAATCTGCCATTCAAGGCTCTAATGT |
| | TCCTAACCCTGATGAGGCCGTCCCTAGTTTTGTTTC |
| | TGGTGCTATGGCTAAAGCTGGTAAAGGACTTCTTGA |
| | AGGTACGTTGCAGGCTGGCACTTCTGCCGTTTCTGA |
| | TAAGTTGCTTGATTTGGTTGGACTTGGTGGCAAGTC |
| | TGCCGCTGATAAAGGAAAGGATACTCGTGATTATCT |
| | TGCTGCTGCATTTCCTGAGCTTAATGCTTGGGAGCG |
| | TGCTGGTGCTGATGCTTCCTCTGCTGGTATGGTTGA |
| | CGCCGGATTTGAGAATCAAAAAGAGCTTACTAAAAT |
| | GCAACTGGACAATCAGAAAGAGATTGCCGAGATGCA |
| | AAATGAGACTCAAAAAGAGATTGCTGGCATTCAGTC |
| | GGCGACTTCACGCCAGAATACGAAAGACCAGGTATA |
| | TGCACAAAATGAGATGCTTGCTTATCAACAGAAGGA |
| | GTCTACTGCTCGCGTTGCGTCTATTATGGAAAACAC |
| | CAATCTTTCCAAGCAACAGCAGGTTTCCGAGATTAT |
| | GCGCCAAATGCTTACTCAAGCTCAAACGGCTGGTCA |
| | GTATTTTACCAATGACCAAATCAAAGAAATGACTCG |
| | CAAGGTTAGTGCTGAGGTTGACTTAGTTCATCAGCA |
| | AACGCAGAATCAGCGGTATGGCTCTTCTCATATTGG |
| | CGCTACTGCAAAGGATATTTCTAATGTCGTCACTGA |
| | TGCTGCTTCTGGTGTGGTTGATATTTTTCATGGTAT |
| | TGATAAAGCTGTTGCCGATACTTGGAACAATTTCTG |
| | GAAAGACGGTAAAGCTGATGGTATTGGCTCTAATTT |
| | GTCTAGGAAATAACCGTCAGGATTGACACCCTCCCA |
| | ATTGTATGTTTTCATGCCTCCAAATCTTGGAGGCTT |
| | TTTTATGGTTCGTTCTTATTACCCTTCTGAATGTCA |
| | CGCTGATTATTTTGACTTTGAGCGTATCGAGGCTCT |
| | TAAACCTGCTATTGAGGCTTGTGGCATTTCTACTCT |
| | TTCTCAATCCCCAATGCTTGGCTTCCATAAGCAGAT |
| | GGATAACCGCATCAAGCTCTTGGAAGAGATTCTGTC |
| | TTTTCGTATGCAGGGCGTTGAGTTCGATAATGGTGA |
| | TATGTATGTTGACGGCCATAAGGCTGCTTCTGACGT |
| | TCGTGATGAGTTTGTATCTGTTACTGAGAAGTTAAT |
| | GGATGAATTGGCACAATGCTACAATGTGCTCCCCCA |
| | ACTTGATATTAATAACACTATAGACCACCGCCCCGA |
| | AGGGGACGAAAAATGGTTTTTAGAGAACGAGAAGAC |
| | GGTTACGCAGTTTTGCCGCAG |
| | AGCTGGCTGCTGAACGCCCTCTTAA |
| | GATATTCGCGATGAGTATAATTACCCCAAAAAGAA |
| | AGGTATTAAGGATGAGTGTTCAAGA |
| | TTGCTGGAGGCCTCCACTATGAAATCGCGTAGAGGC |
| | TTTGCTATTCAGCGTTTGATGAATGCAATGCGACAG |
| | GCTCATGCTGATGGTTGGTTTATCGTTTTTGACACT |
| | CTCACGTTGGCTGACGACCGATTAGAGGCGTTTTAT |
| | GATAATCCCAATGCTTTGCGTGACTATTTTCGTGAT |
| | ATTGGTCGTATGGTTCTTGCTGCCGAGGGTCGCAAG |
| | GCTAATGATTCACACGCCGACTGCTATCAGTATTTT |
| | TGTGTGCCTGAGTATGGTACAGCTAATGGCCGTCTT |
| | CATTTCCATGCGGTGCACTTTATGCGGACACTTCCT |
| | ACAGGTAGCGTTGACCCTAATTTTGGTCGTCGGGTA |
| | CGCAATCGCCGCCAGTTAAATAGCTTGCAAAATACG |
| | TGGCCTTATGGTTACAGTATGCCCATCGCAGTTCGC |
| | TACACGCAGGACGCTTTTTCACGTTCTGGTTGGTTG |
| | TGGCCTGTTGATGCTAAAGGTGAGCCGCTTAAAGCT |
| | ACCAGTTATATGGCTGTTGGTTTCTATGTGGCTAAA |
| | TACGTTAACAAAAAGTCAGATATGGACCTTGCTGCT |
| | AAAGGTCTAGGAGCTAAAGAATGGAACAACTCACTA |
| | AAAACCAAGCTGTCGCTACTTCCCAAGAAGCTGTTC |
| | AGAATCAGAATGAGCCGCAACTTCGGGATGAAAATG |
| | CTCACAATGACAAATCTGTCCACGGAGTGCTTAATC |
| | CAACTTACCAAGCTGGGTTACGACGCGACGCCGTTC |
| | AACCAGATATTGAAGCAGAACGCAAAAAGAGAGATG |
| | AGATTGAGGCTGGGAAAAGTTACTGTAGCCGACGTT |
| | TTGGCGGCGCAACCTGTGACGACAAATCTGCTCAAA |
| | TTTATGCGCGCTTCGATAAAAATGATTGGCGTATCC |
| | AA |

The staple sequences are provided in Table 2 below (as SEQ ID NOs 2 to 109):

| Name | Sequence |
|---|---|
| 20nm4mertip- | GTTCAGAAAACTGGCCTAACCACTGCAACAAG |
| 01 | AAAA |
| 20nm4mertip- | GCGTCACCTGAAACAATAAGAGGTTTTGAAGA |
| 02 | AATAACATCATGGTAACG |
| 20nm4mertip- | AAAGCGGCATAGATATTCAAATAAGTACGGTC |
| 03 | AGGCATCC |
| 20nm4mertip- | AGGCGCTGAACGGACTTCATAGAAGCGC |
| 04 | |
| 20nm4mertip- | TATAACGTGCTTTAGGTGTCTGTAGAACCAGC |
| 05 | AATAAAAG |
| 20nm4mertip- | CTGCATGAATATCAGCACCAACAGATTAGCGG |
| 06 | CGTTGACATATCAAAA |
| 20nm4mertip- | ACGGCGCTTTACCAAATACCTAAATAGTTGTT |
| 07 | ATGGTC |
| 20nm4mertip- | TTAAACTCAAAATTTTACATTAAAATCATGGT |
| 08 | |
| 20nm4mertip- | AATATGACGGTAAAGAACCAGTAGTGTCATAG |
| 09 | CCAGATGCCCGTCA |
| 20nm4mertip- | ATAACGATACCACTGACCCTAATCACCATGGT |
| 10 | |
| 20nm4mertip- | AGAAAAACCGGTCGGACCACCATTACCATCCA |
| 11 | AAGGATAAACCCAC |
| 20nm4mertip- | TCTGAATGGGGTCGGCATCAAAAGTAATCACG |
| 12 | TTCTTGGT |
| 20nm4mertip- | ACCAAAAACCATTTTTGAATCTCTCAGA |
| 13 | |
| 20nm4mertip- | CAGCAAGACAATATCACGAAAATATAATCGGT |
| 14 | AACCAACC |
| 20nm4mertip- | CAGTATGCAAATTAGCACAGGCAAAAAATTTA |
| 15 | CAATGA |
| 20nm4mertip- | CATCACACAGTCCTTGACGGTCGTTCTCTAAT |
| 16 | CCGC |
| 20nm4mertip- | CTGTCGCACCTCCAGCCGGCAAAAGTGGTCTA |
| 17 | |
| 20nm4mertip- | ACTCAGAAGAGAAATAAGCGAACCAAATAAGC |
| 18 | AGCTTGCAGACCCATAAT |
| 20nm4mertip- | AGCAGCCTTATGGCCGTCAAGGAGCACATCGG |
| 19 | |
| 20nm4mertip- | GTCAATAGTAAAGTGCACCGCATGCGGCCATT |
| 20 | AGCTGTACACCCTCGG |
| 20nm4mertip- | TACCCTTGAAAGTGGGACGACCAAAAAGTCTC |
| 21 | AGGAGGAAGCGGAGCAGT |
| 20nm4mertip- | TGCGGTGAAAAAGCGTCCTGAGCGCGCATAAA |
| 22 | CGTA |
| 20nm4mertip- | CACACAAACTGTAGGAAGTGTCCGGTGGTAGA |
| 23 | AGTCGTCA |
| 20nm4mertip- | GATAGGTCGTAGTAATGGATACGCGCGCCGCC |
| 24 | |
| 20nm4mertip- | GATAGCGGCGACTGGCAGTCGGCGTGACTGTA |
| 25 | ACCATAAGGCGAAC |
| 20nm4mertip- | TCTAAACCGAACGTGCCAAGCATAGACAGAAT |
| 26 | AGCTTCTC |
| 20nm4mertip- | TGGTAAGTTGGATTAAGCACCCCAGCCTCACA |
| 27 | |
| 20nm4mertip- | TTTGGCGAGAAAGCTCTTAGGGTCAACGCTAC |
| 28 | AATACT |
| 20nm4mertip- | TGTTAATTTGAGCAGACTCATTCTAGCT |
| 29 | |
| 20nm4mertip- | CCAAATGTTGATTTCTTACCTATTCAGCATCG |
| 30 | GACTCAGAGACTCATA |
| 20nm4mertip- | GTTAGCCTCTGAAACAAATGCTTATGGTATCA |
| 31 | GGGTTAATGTGGCATT |
| 20nm4mertip- | GTCAAATGAGCCTGACAAGAGAATCTATCGAA |
| 32 | ATCATCTTCGTGTT |
| 20nm4mertip- | TTTCTTCTGCGTCAGTAAGAACCAGGGCCTTT |
| 33 | |
| 20nm4mertip- | GGAAACCATAACGAGCTGGAAACGTACGGATT |
| 34 | TAAAAT |
| 20nm4mertip- | ACGGCGTCACCAATCTGCCGAAGCTTGCCTTT |
| 35 | |
| 20nm4mertip- | CGCTCTTTGTTCAGTAACTTGACTTTGAGATG |
| 36 | GCAGCAAC |
| 20nm4mertip- | AGAGGTTTATGGGATCCAAAGCGGTCATCATC |
| 37 | TTGATTAAGCTCATTAGG |
| 20nm4mertip- | AACACCATCTTAATCCACTGTTCAATGTCTAC |
| 38 | GAGAAAGA |
| 20nm4mertip- | CGGCAACATACCAAAGGCGGCTTTACGT |
| 39 | |
| 20nm4mertip- | ACTCAGCGGTCAGTAGCAATGTTGACCACCTG |
| 40 | CAAT |
| 4T5′chl-01 | CAGCAAGGTAGCGACAGTTGTTCCTTGAACGG |
| | CGTCGCGTCTGC |
| 4T5′chl-02 | AGCACCAAACTTGTTAAATCCCAGCG |
| 4T5′chl-03 | GTACTGAATACAAAACTCGGTATAGATAAGCA |
| | GGAGAAACAAGC |
| 4T5′chl-04 | CAACAGGCGCGAATATCTTTTTGGATTCTTTA |
| 4T5′chl-05 | GCGCGAGCGCCAAAATGGTA |
| 4T5′chl-06 | TCTTTAATCCAGCAATCACCTCACCAGATACA |
| | AACTCATCATTA |
| 4T5′chl-07 | GAACATGGGCATATTATCATAAAACGCCCACG |
| | CAAAAAGCGGCT |
| 4T5′chl-08 | GAAGATTTCACGCGGCGGCAAGTTGCCATCTC |
| | TTTAAAGAAGGTCAT |
| 4T5′chl-09 | GTACGGGGTGAATCGCAATCTTTTTTAAGTGG |
| 4T5′chl-10 | AAGTGACGTTTGAGAGATTACCGCGC |
| 4T5′chl-11 | ACGAAGTAGCGGTAAAGTAGCCTCT |
| 4T5′chl-12 | CTTCGTCGCAGTCGAACAAGCGGCGCCATCTG |
| | TTGACAGG |
| 4T5′chl-13 | TTTATAGGGTTTGAATTCATGCGGAGTCAAAG |
| 4T5′chl-14 | AGTCATGGCGACCTGGAGTAACAGAAGTACAG |
| | GTGCCATAAACA |
| 4T5′chl-15 | ACACGAAGGCGGCTCAGCGG |
| 4T5′chl-16 | TCCGACAGGAGCAGGAAACGGAGTA |
| 4T5′chl-17 | ACTTTCAGCGAAGGCATTTAGTCATGATAAGG |
| | ACGTGAAA |
| 4T5′chl-18 | TTCATGACTTTTTTTAGCCATACTCATCCACA |
| | ACCAGTAG |
| 4T5′chl-19 | CGGGATGAAATGTTTTTTCCATGACCTTCTGC |
| | ACGTAATTTAGA |
| 4T5′chl-20 | GCGACGTGTAGCCACGTATTCGCCAG |
| 4T5′chl-21 | GGTTCAATCATTTTTATCGAAAGGTCGCTCAT |
| 4T5′chl-22 | ACGCTCGCAAGAGTAAACTTGGTCA |
| 4T5′chl-23 | AATTACATAGAAACCAACCACTTCG |
| 4T5′chl-24 | GACCCATCAACAGGGACATAAAAAGTAAATAA |
| | ACGTAAGAAACG |
| 4T5′chl-25 | ACGCAACCGGACGCTCGACGCCATTAATACAT |
| | AATAACATCACTATA |
| 4T5′chl-26 | CCTCAACGACTTCTGCCATCAGAATGAGACAG |
| 4T5′chl-27 | CGAGGTCAGAAATCCAACGCGTCAGTTTAAGC |
| | CACTCAGCGTAC |
| 4T5′chl-28 | TGAGTATAATAAATCATAGGCTGAAT |
| 4T5′chl-29 | ACCCGATTCTGAACAGCTTCTTGGGAAGTCCA |
| | TATCATATCTGGGGT |
| 4T5′chl-30 | CATTAGCAATGAAAACTCAAAAGTGTTACAGC |
| | GACGACAA |
| 4T5′chl-31 | CCCTTTGTAGCAGATTTCAT |
| 4T5′chl-32 | TCGTCAATCTCAAATTCGTA |
| 4T5′chl-33 | GGCGCTGCGTAACCGTCTTCAGTGTCAAATCA |
| 4T5′chl-34 | ACGTTCCAAGAGCTCAGAAGCAATACCGAACC |
| | TGATCTCAGTAAATC |
| 4T5′chl-35 | CATATTTAACCTGACTATTCTTGAATTAAAAA |
| 4T5′chl-36 | ACGCCCCTGCAATTAAAATTAGGCCACGAGGA |
| 4T3′chl-01 | ACCCTTCCTGAATGAATGGGATAC |
| 4T3′chl-02 | ACGCTTGTGCCAATTCATCCACGA |
| 4T3′chl-03 | CAGTCAGCCATATAACTGACCA |
| 4T3′chl-04 | AGTGGAGGTTGCATTCAAACGATACGTCAGCC |
| | AACG |
| 4T3′chl-05 | AGCAATAGACCAAACCATCAAT |
| 4T3′chl-06 | TTCGCATAGTGCCATGCTACAC |
| 4T3′chl-07 | GTCGCAAATGAACCTTCAACGGCAGAAGCTTA |
| | AT |
| 4T3′chl-08 | AACGCAGACGACACCATTCGGGAGGGTAAAGA |
| | AG |
| 4T3′chl-09 | AACAAGCAGAATTTTCAAAGTAAGCGTTAGTT |
| | GATG |
| 4T3′chl-10 | AAGTTGGGCATACATACAACGCCCTGCATACG |
| | AAAAGACACGTC |
| 4T3′chl-11 | TTTCTTGTTGACAGTCCCAAGCTATTTAATTG |
| | CG |
| 4T3′chl-12 | CAAAAATTCTAAGCAGTGGCGAGATTATCAGA |
| | AAAA |
| 4T3′chl-13 | TTCTTGCACAGCAATCAATCACCAGAACGGAA |
| | AACATCCTGGAA |
| 4T3′chl-14 | CCTAGAGATGTATGACGCGCATGACAAGTTGT |
| | CA |
| 4T3′chl-15 | CCAACGAAGAAGCAGCATTAACCGTCAATGTA |
| | TCCA |
| 4T3′chl-16 | CTTTGCATTGGGTGAATCATTAGCCTTGTACT |
| | CAGG |
| 4T3′chl-17 | AGTCTCTCCTCACTACCATGAACAAAATGTAA |
| | TCCA |
| 4T3′chl-18 | ATTTTCTCTCTTTTTGCGTTCGTA |
| 4T3′chl-19 | ATAAGACGCATCTCGAACGCAATGAGTAGAGT |
| | CAAT |
| 4T3′chl-20 | TAACTTTTTCCGTGGAAGCATTTTCATCCCGA |
| | AGTTGCGGTTTG |
| 4T3′chl-21 | TGCGGACGACGTCAGTCGCAAGGTAAACGCGA |
| | ACAATTCAACGA |
| 4T3′chl-22 | GTTGGAACGTTTTTTACCTTTTTG |
| 4T3′chl-23 | ATAACGCGAGGGTATCCTAGCA |
| 4T3′chl-24 | AACAGACGATGATTAACAGTCGGGAGAGTGCC |
| | AAGA |
| Trigger01 | CCGCTTCGGCGTTATAACCTCACAC |
| Trigger02 | CATGGAAGCGATAAAACTCTGCAGG |
| Trigger03 | TCTTGAACACTCATCCTTAATACCT |
| Trigger04 | CTGCTTTATCAAGATAATTTTTCGA |
| Trigger05 | TTAAGAGGGCGTTCAGCAGCCAGCT |
| Trigger06 | TAGACATAATTTATCCTCAAGTAAG |
| Trigger07 | GTGGTCGGCAGATTGCGATAAACGG |
| Trigger08 | CCAGCAAGGAAGCCAAGATGGGAAA |
| Analyte | CATCACACAGTCCTTGACGGTCGTTCTCTAAT |
| strand | CCGC |

Single-channel current recordings were carried out using an integrated chip-based, parallel recording setup (Orbit Mini and Orbit 16, Nanion Technologies, Munich, Germany) with multielectrode-cavity-array (MECA) chips (IONERA, Freiburg, Germany)(33, 35). Bilayers were formed of 1,2-Diphytanoyl-sn-Glycero-3-Phosphatidylcholine (DPhPC) dissolved in octane to a final concentration of 10 mg/mL. Electrophysiological buffer was composed of 1M KCl, 10 mM HEPES, pH 7.4. For pore insertions, Square nanomechanical pores were mixed in a 2:1 (vol/vol) ratio with 0.5% OPOE (n-octyloligooxyethylene) in 1 M KCl, 10 mM HEPES, PH 7.4.

The mixture was applied to the cis chamber and insertions monitored by increases in conductance steps. The current traces were not Bessel-filtered and acquired at 10 kHz using Element Data Recorder software (Element s.r.l., Italy).

For analyte sensing experiments, a single stranded nucleic acid sequence was used as the analyte. The analyte strand is SEQ ID NO: 170 (see Table 2) which is complementary in sequence to the trigger region and capable of hybridising to it such that the trigger region assumes a more rigid double helix structure. Consequently, upon analyte binding the nanostructure transitions to second conformation (see FIG. 2). The analyte was diluted in electrophysiological buffer to the desired concentration. Upon successful nanopore insertion, diluted analyte was added to the cis chamber. The Orbit 16 was used for analyte sensing experiments; the Orbit mini was used in all other electrophysiological experiments. The Orbit 16 and Orbit mini are grounded at the cis and trans, respectively. To aid comparison, voltages were normalized and are presented as positive in relation to the cis chamber. Single-channel analysis was performed using Clampfit software (Molecular Devices, Sunnyvale, CA, USA).

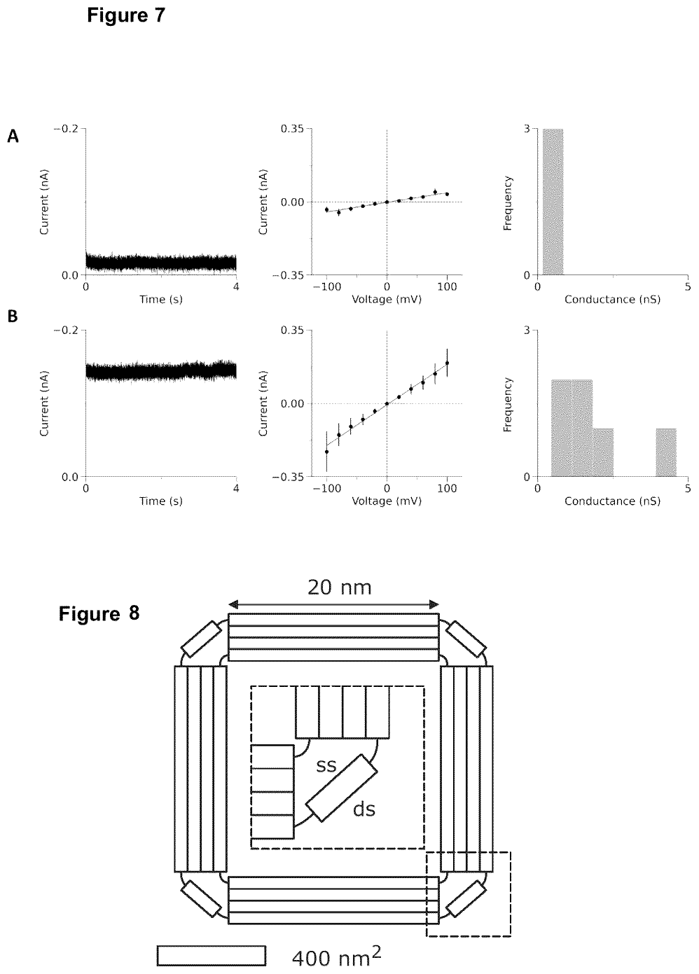

The results are shown in FIG. 7 which shows analysis with current recording. The lower conductance recording (FIG. 7A) corresponds to collapsed/un-expanded configuration in absence of analyte (i.e. FIG. 2A). The level of current passing through the pore is low, just above zero (left hand and middle traces), because the pore lumen is almost closed. In the presence of analyte the pore transitions to the expanded state (i.e. FIG. 2B). This translates to a higher conductance state of the pore (FIG. 7B).

The results demonstrate that a nanomechanical change in conformation in response to binding of an analyte can result in a significant and detectable change current passing through the pore.

The mechanism demonstrated in this invention is quite distinct from conventional nanopore biosensing which relies upon an analyte obstructing the lumen of the pore to effect detectable current blockade. In the present example the analyte is a nucleic acid that hybridises to the pore structure and causes a conformational change. In this instance the analyte does not obstruct the lumen of the pore at all and yet a readily detectable change in current results in the presence of analyte due to the unique mechanism of action.

### Example 2—Detection of Protein Analytes

To prepare a protein-sensitive nanomechanical pore, a pore design illustrated in FIG. 9A-C was developed. In the design, the pore exists in two conformations: a closed conformation of oblique shape and limited lumenal cross sectional area. The pore was selected to have a 10 nm side length, thereby providing a maximal cross section lumenal surface area of 100 nm² when the pore is the second conformation, and a smaller surface area when in the first conformation.

To render the pore sensitive to proteins, a protein receptor is positioned at one or two vertices, or corners, of the nanopore, shown for the M1 and M2 pore variants (FIG. 9A, 9B, pore the left side of the panel, the ligand is shown as a dark circle). In the absence of a bound protein analyte, the nanomechanical pores remains in the closed state of oblique shape. By comparison, binding of one or two of the globular protein analyte molecules to the receptors of the M1 and M2 pore, respectively, switches the conformation into the open state (FIG. 9B, 9C, right). The reason for the switch lies in the steric bulk of the protein and its influence on the conformation. By comparison, pore variant M0 without a protein receptor, remains in the closed state in the presence of protein analyte (FIG. 9A).

The principle of the protein-sensitive nanomechanical was demonstrated with receptors composed of a biotin tag, and a cognate streptavidin protein analyte molecules. The protein-sensitive nanomechanical pore M2 was prepared by mixing the phix174 scaffold (see Table 1; SEQ ID NO: 1) with the staple strands listed in Table 3 below (SEQ ID NOs: 110 to 169). For nanomechanical pore M0 the mix excluded staple strand 10nmMechPro-57bio (SEQ ID NO: 156) and 10nmMechPro-58bio (SEQ ID NO: 157), while for the pore M1 strand 10nmMechPro-58bio (SEQ ID NO: 157) was excluded. The assembly and purification conditions were analogous to the DNA-sensitive nanomechanical pore described above in Example 1.

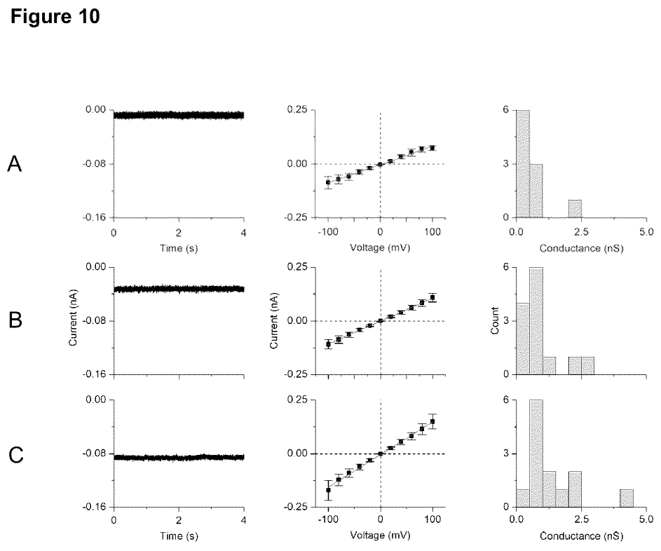

To demonstrate the protein-induced switch of the protein-sensitive nanomechanical pore from the closed to the open state, nanopore variants M0, M1 and M2 were inserted into lipid bilayers and electrophysiologically characterised using the electrical recording OrbinMini devices, as described for the DNA-sensitive pore in Example 1. The single-channel current trace of the pore variants M0 after addition of the streptavidin protein analyte (1 uL from a 5 uM stock solution) to the cis chamber of the recording equipment is shown in FIG. 10A. The corresponding IV curve and conductance histograms are displayed in FIG. 10A. The data indicate a low conductance state, consistent with the a closed conformation of oblique shape and limited lumenal cross sectional area expected for the nanomechanical pore M0 (FIG. 9A). By comparison, adding of the protein analyte to pore variants M1 and M2, led to current traces, IV curves and conductance histograms, shown in FIGS. 10B and 10C, respectively. The data for the M1 and M2 pores indicate a higher conductance state, consistent with the open square pore conformations (FIGS. 9B, 9C).

Hence, the experiment shows that a detectable nanomechanical actuation of a nucleic acid nanopore can be initiated by a stimulus that comprises a protein binding event.

The staple sequences for the protein sensing nanomechanical nanopore are provided in Table 3 below (as SEQ ID NOs: 110 to 169):

| Name | Sequence |
|---|---|
| 10nmMechPro- | TTATCAGATCTGTAAAACAGGTGCGATGTAGCA |
| 01 | ATTAAAA |
| 10nmMechPro- | CATCAGGGTCCTTTACCAGCTTTAACGTACCTA |
| 02 | TTCAGCG |
| 10nmMechPro- | TCTCTTTAGCCAGCAATATCGGTACATACAAAC |
| 03 | CAGAAAA |
| 10nmMechPro- | TGGTATCAGTTGTTATAGATATTCACGGCGCTT |
| 04 | GTTCACC |
| 10nmMechPro- | ATAAATCATATTGTTATCGGTAGCTCACCCAT |
| 05 | |
| 10nmMechPro- | ATAACTTAACCTCAAAGTCTGAAACCACCAAAC |
| 06 | |
| 10nmMechPro- | GCCTACAGTTAGGAACATTAGAGCGACGGCCT |
| 07 | |
| 10nmMechPro- | AACAGTCGGGGTTAATCGTGCCAAGATTTTAT |
| 08 | |
| 10nmMechPro- | CAGAGGAGTGGAGATTTAATACCAGGCACATCA |
| 09 | |
| 10nmMechPro- | CCTTGAATGCCACCGGAGGCGGCTGTAGTGTT |
| 10 | |
| 10nmMechPro- | CTCCAAACAATTTAGACATGGCGCGTGCCATG |
| 11 | |
| 10nmMechPro- | CTCAGGAAGTCGCAGTAGGCGGAAGTACTGAA |
| 12 | |
| 10nmMechPro- | CATGAAACGCAAAGGTCAATATAACTTGACCGC |
| 13 | |
| 10nmMechPro- | GCGCACCTAAGAAAAAGAGTAAACACCAGCAAG |
| 14 | |
| 10nmMechPro- | AGCAGAAGCAATACCGCCAGCAATGATTAAAC |
| 15 | |
| 10nmMechPro- | TCCTAAGCAAAAAAGTTTGAATTAGAACCAGC |
| 16 | |
| 10nmMechPro- | TCAAAATACATCTGAAACCATTACACAACCTG |
| 17 | tcatcttcacactact |
| 10nmMechPro- | AAAATTTAGGGTACAGTCCTCATA |
| 18 | tcatcttcacactact |
| 10nmMechPro- | GATGTATCCGGCATCACAACAGAAGTCGGGAGG |
| 19 | GTAGTCGATAA tcatcttcacactact |
| 10nmMechPro- | ATAATAACGTTGAAGAAGCTCAGCATTA |
| 20 | tcatcttcacactact |
| 10nmMechPro- | ACATTGACGGTAGGACGTCAATAGTCACTCTCT |
| 21 | TTAAAAG tcatcttcacactact |
| 10nmMechPro- | GGCAACCAAACCATGAAACCGTAG |
| 22 | tcatcttcacactact |
| 10nmMechPro- | CAGAAGTGGCGGCAGAACCAAATCCAAGATAA |
| 23 | tcatcttcacactact |
| 10nmMechPro- | AGTTTGAGCACGTCCATCTCACCA |
| 24 | tcatcttcacactact |
| 10nmMechPro- | CTTTATCACTCCCAAGTGCAGCAGGACCAGCAA |
| 25 | GGAAGCCATAA tcatcttcacactact |
| 10nmMechPro- | CCTTCCAGCCTGCATAGCACAAGCAACT |
| 26 | tcatcttcacactact |
| 10nmMechPro- | GACGGAAGGAGTGAGAGCGCCAACGGCGCATGA |
| 27 | ACTGTAA tcatcttcacactact |
| 10nmMechPro- | ATCTGTCTACAGTAGAGTCACATA |
| 28 | tcatcttcacactact |
| 10nmMechPro- | GGTAGAAGTCGTGTTTTACCTTCT |
| 29 | tcatcttcacactact |
| 10nmMechPro- | CGGTACGGCAACGGAATCTTGATTGCGGAGCA |
| 30 | tcatcttcacactact |
| 10nmMechPro- | GATGGCAGCATTTGGCAGGAGGAACAGTATGCA |
| 31 | AATTAGCTAAT tcatcttcacactact |
| 10nmMechPro- | TATATCAGGCATATAACCCTAAGCTCAT |
| 32 | tcatcttcacactact |
| 10nmMechPro- | CACGTCCAAATGGTCAGCGTCATAAGAGGGTCT |
| 33 | GTTTCAA tcatcttcacactact |
| 10nmMechPro- | TGGTATAGCCAGATGCCCAGCAGT |
| 34 | tcatcttcacactact |
| 10nmMechPro- | CACGCGGCTTAAACTTGAACGGAAGCGCATAA |
| 35 | tcatcttcacactact |
| 10nmMechPro- | TGGGAAGCCTTCAATTTAACATCA |
| 36 | tcatcttcacactact |
| 10nmMechPro- | CAGCAATCAAGAAGGTATACGAAGGGCGAATAA |
| 37 | GTACGCGTGGT tcatcttcacactact |
| 10nmMechPro- | GTCAGGCAAGTTAGTCAAAGAACATCCT |
| 38 | tcatcttcacactact |
| 10nmMechPro- | CATACTGACTATATAAACGGTCACATTAACCCC |
| 39 | TCACACT tcatcttcacactact |
| 10nmMechPro- | TGGTCTTTTATCACGAAGTCTAAC |
| 40 | tcatcttcacactact |
| 10nmMechPro- | cctacgagtcagtcct TCCAAAGGGAACCGAA |
| 41 | CCAAACAGGCAA |
| 10nmMechPro- | cctacgagtcagtcct TTAGGGATGGAGGTGG |
| 42 | CGTAACAGAAGTGCGAGAA |
| 10nmMechPro- | cctacgagtcagtcct CGGTATAACCTGTCAA |
| 43 | GAAGACAA |
| 10nmMechPro- | cctacgagtcagtcct GGCATTTAAACATAAA |
| 44 | ACGGGGAATAATAACC |
| 10nmMechPro- | cctacgagtcagtcct AAATGGTAAAGATGGG |
| 45 | ACGCTCGGCGCC |
| 10nmMechPro- | cctacgagtcagtcct AAATCAGTAACAGGCA |
| 46 | TAAACAAAACTATGAATGG |
| 10nmMechPro- | cctacgagtcagtcct AAAATAATCCACTTAA |
| 47 | AATACCAT |
| 10nmMechPro- | cctacgagtcagtcct ATGAGGGAATAGCAAG |
| 48 | GAAAGACGCGCCAGCG |
| 10nmMechPro- | cctacgagtcagtcct GCATGAAGATAAGCAG |
| 49 | GTCAATAGATGT |
| 10nmMechPro- | cctacgagtcagtcct CATCGAACAGAACGGC |
| 50 | AACAAATGCTTAAAAGCGG |
| 10nmMechPro- | cctacgagtcagtcct TCCAGAACACGAACAG |
| 51 | GGTCTGTT |
| 10nmMechPro- | cctacgagtcagtcct GGAAACACTTCTTGCA |
| 52 | TTCCTGAATGAA |
| 10nmMechPro- | cctacgagtcagtcct CAATCACCTTCGCCGC |
| 53 | GCTTTAGCGTTACGAACAA |
| 10nmMechPro- | cctacgagtcagtcct CTCAGCGGCAAATTTA |
| 54 | GGTGCTTG |
| 10nmMechPro- | cctacgagtcagtcct CGTTTGGTAGATTAGA |
| 55 | GGACGGTTAAGAAATA |
| 10nmMechPro- | cctacgagtcagtcct CGGCGTTAATGATTGA |
| 56 | AGATTGCGTCCACTGC |
| 10nmMechPro- | biotin-t |
| 57bio | CGTTTGGTAGATTAGAGGACGGTTAAGAAATA |
| 10nmMechPro- | biotin-t |
| 58bio | CGGCGTTAATGATTGAAGATTGCGTCCACTGC |
| 10nmMechPro- | GGACTGACTCGTAGG-chl |
| 59chl | |
| 10nmMechPro- | chl-AGTAGTGTGAAGATG |
| 60chl | |

Biotin=biotin label that acts as the protein binding moiety—e.g. the receptor
Chl=cholesterol membrane anchor

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, such as the scaffold strand of interest, is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1. A membrane-spanning actuatable nucleic acid nanopore, the nanopore comprising:
one or more polynucleotide strands that provide a scaffold component and a plurality of polynucleotide strands that provide a plurality of staple components wherein each of the plurality of staple components hybridise to the scaffold component; and
a trigger that is actuatable in response to a stimulus, wherein the trigger comprises a linker sequence, and the linker sequence comprises a binding moiety, further wherein the trigger is actuated by a stimulus that comprises binding of an analyte molecule to the binding moiety;
wherein the nanopore defines a channel, and wherein the channel defines a lumen that has a polygonal shape in cross section;
wherein actuation of the trigger results in a conformational change of the nanopore from a first conformation to at least a second conformation, wherein the conformational change results in a change in the shape of the lumen from a first shape to a second shape, and wherein the conformational change is detectable; and
wherein the conformational change results in a detectable change in an electrical signal.

2. The membrane-spanning nanopore of claim 1, wherein either or both of the one or more polynucleotide strands that provide a scaffold component and at least one of the plurality of staple components further comprise at least one hydrophobic anchor that facilitates insertion of the nanopore into the membrane.

3-5. (canceled)

6. The membrane-spanning nanopore of claim 1, wherein the conformational change of the nanopore from a first conformation to at least a second conformation results in a change in the cross-sectional area (CSA) of the lumen from a first lumen CSA to a second lumen CSA.

7. The membrane-spanning nanopore of claim 6, wherein the nanopore comprises one or more nanostructural modules.

8. The membrane-spanning nanopore of claim 7, wherein the nanopore further comprises at least one sub-module connected between the one or more nanostructural modules.

9. The membrane-spanning nanopore of claim 8, wherein the or each module are the same such that the nanopore has a rotational symmetry about the longitudinal axis of the nanopore when the nanopore is in the first conformation or the second conformation.

10. The membrane-spanning nanopore of claim 9, wherein the or each module is connected to at least one other module.

11. The membrane-spanning nanopore of claim 10, wherein the connection between modules is provided by structures selected from the group consisting of: a staple strand or portion thereof of one of the modules; a scaffold strand or portion thereof of one of the modules; a sub-module; one or more polynucleotide strands that provide a spacer component.

12. The membrane-spanning nanopore of claim 11, wherein the at least one hydrophobic anchor is comprised within the one or more nanostructural modules.

13. (canceled)

14. The membrane spanning nanopore of claim 12, wherein the least one nanostructural module that comprises a plurality of hydrophobic anchors attached thereto is configured to be oriented coaxially to the planar surface of a semi-fluid membrane such that the plurality of hydrophobic anchors insert perpendicularly into the semi-fluid membrane.

15. The membrane spanning nanopore of claim 14 wherein the hydrophobic anchor comprises a molecule is selected from the group consisting of: a lipid; and a porphyrin.

16-20. (canceled)

21. The membrane-spanning nanopore of claim 15, wherein the trigger is comprised within the sub-module, further wherein the trigger is located proximate to at least one vertex within the nanopore.

22. The membrane-spanning nanopore of claim 15, wherein the trigger is comprised within the sub-module, further wherein the trigger is located proximate to at least two vertices within the nanopore.

23. The membrane-spanning nanopore of claim 15, wherein the trigger is comprised within the sub-module, further wherein the trigger is located proximate to at least two opposing vertices within the nanopore.

24. The membrane-spanning nanopore of claim 23, wherein the trigger comprises one or more regions of scaffold component that are single stranded.

25. The membrane-spanning nanopore of claim 23, wherein the trigger comprises one or more regions of a staple component that are single stranded.

26. The membrane-spanning nanopore of claim 25, wherein the trigger is actuated by a stimulus that comprises hybridisation of a target oligonucleotide or polynucleotide, or a portion thereof, to a single stranded region of the trigger.

27-28. (canceled)

29. The membrane-spanning nanopore of claim 1, wherein the binding moiety is selected from one or more of the group consisting of:
I. an enzyme—including a polymerase, a helicase, a gyrase, and a telomerase, as well as nucleic acid binding sub domains or derivatives thereof
II. a synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. a synthetic or naturally derived affinity binding nucleic acids and nucleic acid analogues, including oligonucleotide probes, aptamers and ribozymes;
V. a naturally occurring or synthetic small molecule, including biotin, a drug molecule, fluorophores, metabolites and chemokines;
VI. an antigen or antigenic fragment; and
VII. a signalling molecule and/or polypeptide receptors thereof, including binding domains of receptors, and receptor complexes.

30-35. (canceled)

36. The membrane spanning nanopore of claim 1, wherein the change in an electrical signal comprises a change in an electrical current flowing through or across the nanopore.

37. The membrane spanning nanopore of claim 1, wherein the change in an electrical signal comprises a change in impedance of an electrical current flowing through or across the nanopore.

38. A membrane into which is inserted at least one membrane-spanning nanopore as described in claim 26.

39-52. (canceled)
